SEQUENCE LISTING (12 sequences)

SEQ ID NO: 1 - DNA, 29 bp, Artificial Sequence
  Description of Artificial Sequence Probe
atcctccgctggggactttccagggagga

SEQ ID NO: 2 - DNA, 30 bp, Artificial Sequence
  Description of Artificial Sequence Synthetic oligonucleotide
gatcctccgctctcgactttccagggagga

SEQ ID NO: 3 - DNA, 25 bp, Artificial Sequence
  Description of Artificial Sequence Primer
ttgaattcttagtggttgccagcac

SEQ ID NO: 4 - DNA, 25 bp, Artificial Sequence
  Description of Artificial Sequence Primer
gttcctcagcctcttctccttcctg

SEQ ID NO: 5 - DNA, 25 bp, Artificial Sequence
  Description of Artificial Sequence Primer
tcatctttcaacacgcaggacaggt

SEQ ID NO: 6 - DNA, 24 bp, Artificial Sequence
  Description of Artificial Sequence Primer
tcatcttcaacacgcaggacaggt

SEQ ID NO: 7 - DNA, 22 bp, Artificial Sequence
  Description of Artificial Sequence Primer
ctgcacccacttcccaggcaac

SEQ ID NO: 8 - DNA, 23 bp, Artificial Sequence
  Description of Artificial Sequence Primer
ccccagcccagagacaagataaa

SEQ ID NO: 9 - DNA, 21 bp, Artificial Sequence
  Description of Artificial Sequence Primer
gacatactccaaacctttcca

SEQ ID NO: 10 - DNA, 21 bp, Artificial Sequence
  Description of Artificial Sequence Primer
actgtgaggtaagatggtggc

SEQ ID NO: 11 - DNA, 19 bp, Artificial Sequence
  Description of Artificial Sequence Primer
agccttgggaaacatgcgt

SEQ ID NO: 12 - DNA, 21 bp, Artificial Sequence
  Description of Artificial Sequence Primer
ccctgaacaaaagcatccgat

(19) United States
(12) Patent Application Publication
(10) Pub. No.: US 2004/0001856 A1
(43) Pub. Date: Jan. 1, 2004

(54) Extract of lentinus lepideus and composition comprising the same having immune enhancing activity

(75) Inventors: Mirim Jin, Seoul (KR); Hyung-Jin Jung, Seoul (KR); Sung-Seup Shin, Seoul (KR); Hyang Jeon, Seoul (KR); Jeong-June Choi, Icheon (KR); Sunyoung Kim, Seoul (KR)

Correspondence Address: FOLEY AND LARDNER, SUITE 500, 3000 K STREET NW, WASHINGTON, DC, 20007, US

(73) Assignee: Pangenomics Co., Ltd.

(21) Appl. No.: 10/383,773

(22) Filed: Mar. 10, 2003

Related U.S. Application Data
(60) Provisional application No. 60/362,638, filed on Mar. 8, 2002.
(60) Provisional application No. 60/372,056, filed on Apr. 12, 2002.

(51) Int. Cl. A61K 35/84

(52) U.S. Cl. 424/195.15

(57) ABSTRACT

The present invention is related to pharmaceutical composition comprising an extract of Lentinus lepideus prepared by inventive preparation showing immune enhancing and immune modulating activity.

The composition according to the present invention are useful in the prevention or treatment of infectious diseases caused by bacteria or virus and treatment of immune-suppressed patients, suppressed immunity due to anti-cancer therapy such as chemotherapy and radiotherapy and the patients suffered from AIDS or cancer and immune enhancing agent. Moreover, present composition can be used in preventing bacterial infection or viral disease such as influenza and also can be used for adjuvants of vaccine.

### CROSS REFERENCE TO RELATED APPLICATION

This application is a continuation patent application of U.S. Provisional Application No. 60/362,638 and No. 60/372,056 filed on Mar. 8, 2002 and Apr. 12, 2002, which were now abandoned.

### DESCRIPTION

1. Field of the Invention

The present invention related to an extract of Lentinus lepideus and a composition comprising the same having immune enhancing activity.

2. Background of the Invention

Mycelia or fruit bodies of various mushrooms have been found to contain biological response modifiers (BRMs). Indeed, various extracts from medicinal mushrooms have been reported to exhibit antiviral, antibiotic, anti-inflammatory, hypoglycemic, and hypotensive activities (Kabir Y et al.; J. Nutr. Sci. Vitaminol. (Tokyo), 33, pp341-346, 1987). The most widely known effects of the compounds isolated from above described fungi are anti-tumor and immune modulating activities of BRMs isolated from Ganoderma lucidum, Lentinus edodes, and Grifola frondosa. For example, oral administration of powdered fruit bodies of Lentinus edodes has been found to be effective in inhibiting carcinoma growth in C3H/He mice (Nanba H and Kuroda H, Chem. Pharm. Bull. (Tokyo), 35, pp2459-2464, 1987). It has been reported that the cytotoxic activity of NK (Natural Killer) and LAK (Lymphokine-Activated Killer) cells was significantly increased by administration of Lentinus edodes (Nanba H and Kuroda H, Chem. Pharm. Bull. (Tokyo), 35, pp2459-2464, 1987; Nanba H et al.; Chem. Pharm. Bull. (Tokyo), 35, pp2453-2458, 1987). In administering mice pretreated with immunosuppressive carcinogen, with mushroom-enriched diet comprising Lentinus edodes, Grifola frondosa, and oyster mushroom, the level of the chemotactic activity of macrophage and the capability of lymphocyte to proliferate in response to mitogen were restored to the normal level (Kurashige S et al.; Immunopharmacol. Immunotoxicol., 19, pp175-183, 1997). A number of such bioactive molecules have been reported in numerous other mushroom species.

Polysaccharides, which has mostly branched (1→3)-β-D glucan structure, have been reported to contain potent anti-tumor and immune modulating activity by interacting with various immune cells. Water-soluble glucans such as “lentinan” isolated from Lentinus edodes and “schizophyllan” from Schizophillium commune have been used for cancer immuno-therapy in Japan for more than 15 years (Okamura et al., Biotherapy, 1, pp103-107, 1989; Suto et al., Cancer Chemother. Pharmacol., 33, pp145-148, 1994). These BRMs contain numerous biological activities including induction of hematopoiesis, activation of cytokine system, inhibition of tumor cell growth, and induction of resistance to viral or bacterial infection (Borchers et al., Pro. Soc. Exp. Biol. Med., 221, pp281-293, 1999). Clinical studies have revealed that “lentinan” has been effective in prolonging the survival of patients suffered from cancer, in particular, gastric and colorectal cancer (Nakano et al., Hepatogastroenterology, 46, pp2662-2668, 1999). The activities of LAK and NK cell were significantly increased with i.v. injection of lentinan (Arinaga et al., Int. J. Immunopharmacol., 14, pp535-539, 1992). It has been demonstrated that levels of IL-1 and TNF-α are increased in human macrophage (Fruehauf et al., Life Sci., 64, pp1005-1011, 1999; Takeshita et al., Surg. Oncol., 5, pp23-28, 1996) and further T cell response to IL-2 was increased and LAK and NK cells were activated upon treatment with lentinan (Suzuki et al., Cancer Immunol. Immunother., 28, pp1-8, 1994).

Radiotherapy and/or chemotherapy often result in hematopoietic and immune dysplasia in accordance with the damage of hematopoietic stem cells during the procedure, and hematopoietic and immune cells are depleted to the end. Consequently, patients often suffered from anemia, lymphocytopenia, thrombocytopenia, and/or granulocytopenia, leading to serious and lethal infections and increasing the mortality of these patients. Irradiation affects almost all the subpopulations undergoing cell division, including early blasts present in bone marrow. Therefore, main critical factor of the restoring rate of the patients is the percentage of numbers of remaining resting stem cells during or after radiotherapy and/or chemotherapy.

As means to protect stem cells or help damaged stem cells to recover, the use of biological responsive modifiers (BRMs) has been paid attention to. Various compounds, especially carbohydrates isolated from mushrooms, yeasts and plants, were reported to affect bone marrow and peripheral blood cells and induce hematopoiesis (Hofer M et al., J. Leukoc. Biol., 53, pp185-189, 1993). For example, a single peritoneal injection of Scleroglucan isolated from Sclerotium glucanicum, enhanced the bone marrow cellularity, while OL-2 isolated from Omphalia lapidescens, increased the number of lymphocytes and various immune cells in both peritoneal and spleen organ (Pretus H A et al., J. Pharmacol. Exp. Ther., 257, pp500-510, 1991).

Intravenous injections of glucan-F, water soluble glucan, increased the number of GM-colony forming units as well as the erythroid colony forming unit (Patchen M. L. et al.; J. Immunopharmacol., 8, pp407-425, 1986: Patchen M L. et al.; J. Biol. Response. Mod., 3, pp627-633, 1984: Patchen ML and Macvittie T J; J. Biol. Response. Mod., 5, pp45-60, 1986). The mechanism of these observation is not found clearly till now. However, it has been reported that carbohydrates having hematopoietic activity (Egger S F et al.; Int. J. Immunopharmacol., 18, pp113-126, 1996), such as CARN750 (water soluble β-(1, 4)-linked acetylated mannan), isolated from Aloe barbadenis, could activate macrophage and monocytes, inducing the production of IL-1, IL-6, interferon and GM-CSF (granulocyte-macrophage colony stimulating factor) in vitro experiment (Zhang L and Tizard I R.; Immunopharmacology, 35, pp119-128, 1996) Lentinus lepideus is an edible mushroom belonging to Agaricales of Basidiomycotina and comprises 26 free amino acids, ergosterol, 6 saturated fatty acids, 43 unsaturated fatty acids, 8 trace metal elements, anisic acid methyl, eburicoic acid, lentinamycin A, B, bis-methylsulfonylmethyldisulfide, β-1,3-glucanase and so on and it is known that Lentinus lepideus shows anti-tumor, antibiotic, anti-fungal activity and human body condition-controlling effect. (Park W. H. and Lee H. D., Illustrated Book of Korean Medicinal Mushrooms, KyoHakSa, pp392-393, 1999).

KR 1997-0005304 (A) discloses the immuno-potentiator and its manufacturing method using Aspergillus Oryzae and Lentinus edodes, and KR 2001-0046746 (A) discloses the composition comprising β-1,6-branced-β-1,3-glucan isolated from Schizophillium commune, Sclerotium glucanicum and Lentinus edodes for anti-inflammation and alleviating skin irritation.

To develop the extract of Lentinus lepideus as a BRM, the inventors of the present invention have intensively carried out the scientific investigation concerning the pharmacological effects of a Lentinus lepideus as an immune modulator.

As a result of the investigation, the inventors have finally discovered that the extract of Lentinus lepideus led the production/activation of various cytokines, and the substantial enhancement of immunity.

### SUMMARY OF THE INVENTION

According to one aspect, the present invention provides an extract of Lentinus lepideus showing immune enhancing and immune modulating activity.

The present invention provides a pharmaceutical composition comprising the above extract as an active ingredient in an effective amount to prevent infectious disease and to treat immune-suppressed patients by immune enhancement, together with pharmaceutically acceptable carrier.

The present invention provides a use of above extract for the preparation of pharmaceutical composition to prevent infectious diseases, to treat immune-suppressed patient and to add to adjuvant of vaccine.

The present invention provides a method for treating a mammal or human afflicted with infectious diseases comprising administering to a mammal or human an effective amount of above extract together with a pharmaceutically acceptable carrier thereof.

The present invention also provides a health food comprising above extract as an active ingredient in an effective amount to prevent infectious diseases and enhance immunity together with a sitologically acceptable additive.

### BRIEF DESCRIPTION OF THE DRAWINGS

The above and other objects, features and other advantages of the present invention will more clearly understood from the following detailed description taken in conjunction with the accompanying drawings, in which;

FIG. 1 presents HPLC analysis of monosaccharide moiety of PG101-1 prepared in Example 1;

FIG. 2 shows the effect of PG101-1 on induction of various cytokines according to its concentration in human peripheral blood cell (PBC);

FIG. 3 presents the effect of PG 101-1 on induction of various cytokines with passage of time in human PBC;

FIG. 4 depicts the promoter structures of various cytokines;

FIG. 5 shows the luciferase activity using NF-κB reporter plasmid after PG101-1 treatment;

FIG. 6 presents the binding activity of NF-κB induced by PG101-1 in human mononuclear cell using EMSA (Electro Mobility Shift Assay).

FIG. 7a exhibits the effect of PG101-1 and PDTC on TNF-αinduction;

FIG. 7b presents the effect of PG101-1 and PDTC on IL-1β induction;

FIG. 7c shows the effect of PG101-1 and PDTC on IL-10 induction;

FIG. 7d presents the effect of PG101-1 and PDTC on IL-12 induction;

FIG. 7e represents the effect of PG101-1 and PDTC on GM-CSF induction;

FIG. 7f exhibits the effect of PG101-1 and PDTC on IL-18 induction;

FIG. 8a presents the toxicity of PG101-1 to HT1080;

FIG. 8b presents the toxicity of PG101-1 to U937;

FIG. 9a shows effects of PG101-1 on CFU (colony forming unit) in irradiated mice;

FIG. 9b shows effects of PG101-1 on CFU-GM in irradiated mice;

FIG. 9c shows effects of PG101-1 on BFU-C in irradiated mice;

FIG. 10a represents a two-color FACS analysis using ER-MP12 & ER-MP20 antibodies in normal mice;

FIG. 10b represents effects of PBS on bone marrow cells in irradiated mice;

FIG. 10c represents effects of PG101-1 on bone marrow cells in irradiated mice;

FIG. 10d represents effects of G-CSF on bone marrow cells in irradiated mice;

FIG. 11a represents a dot plot of bone marrow cells on 16^{th} day in normal mice;

FIG. 11b shows effects of PBS on bone marrow cells on 16^{th} day in irradiated mice;

FIG. 11c shows effects of PG101-1 on bone marrow cells on 16^{th} day in irradiated mice;

FIG. 11d shows effects of G-CSF on bone marrow cells on 16^{th} day in irradiated mice;

FIG. 11e represents a histogram using PE-conjugated c-Kit antibodies of bone marrow cells in normal mice;

FIG. 11f shows effects of PBS on c-Kit positive cells on 16^{th} day in irradiated mice;

FIG. 11g shows effects of PG101-1 on c-Kit positive cells on 16^{th} day in irradiated mice;

FIG. 11h shows effects of G-CSF on c-Kit positive cells on 16^{th} day in irradiated mice;

FIG. 11i represents a dot plot of bone marrow cells on 24^{th} day in normal mice;

FIG. 11j shows effects of PBS on bone marrow cells on 24^{th} day in irradiated mice;

FIG. 11k shows effects of PG101-1 on bone marrow cells on 24^{th} day in irradiated mice;

FIG. 11l shows effects of G-CSF on bone marrow cells on 24^{th} day in irradiated mice;

FIG. 11m represents a histogram of bone marrow cells using PE-conjugated c-Kit antibody on 24^{th} day in normal mice;

FIG. 11n shows effects of PBS on c-Kit positive cells on 24^{th} day in irradiated mice;

FIG. 11o shows effects of PG101-1 on c-Kit positive cells on 24^{th} day in irradiated mice;

FIG. 11p shows effects of G-CSF on c-Kit positive cells on 24^{th} day in irradiated mice;

FIG. 11q represents a histogram of bone marrow cells using PE-conjugated Gr-1 antibody on 24^{th} day in normal mice;

FIG. 11r shows effects of PBS on Gr-1 positive cells on 24^{th} day in irradiated mice;

FIG. 11s shows effects of PG101-1 on Gr-1 positive cells on 24^{th} day in irradiated mice;

FIG. 11t shows effects of G-CSF on Gr-1 positive cells on 24^{th} day in irradiated mice;

FIG. 12a shows the effect of PG101-1 on the cytokine, IL-1β;

FIG. 12b shows the effect of PG101-1 on the cytokine, IL-6;

FIG. 12c shows the effect of PG101-1 on the cytokine, GM-CSF;

FIG. 12d shows the effect of PG101-1 on the cytokine, TNF-α;

FIG. 13a presents TNF-α production in PG101-1, PG101-2 and lentinan-treated human PBMCs;

FIG. 13b represents IL-1β production in PG101-1, PG101-2 and lentinan-treated human PBMCs;

FIG. 13c shows the IL-10 production in PG101-1, PG101-2 and lentinan-treated human PBMCs;

FIG. 13d shows IL-12 production in PG101-1, PG101-2 and lentinan-treated human PBMCs;

FIG. 13e shows GM-CSF production in PG101-1, PG101-2 and lentinan-treated human PBMCs;

FIG. 14 depicts the effect of PG101-1 on the cytokine gene expression;

FIG. 15 exhibits the effect of PG101-1 and cyclohexamide on the gene expression of various cytokines.

### DETAILED DESCRIPTION

Accordingly, it is an object of the present invention to provide an extract of Lentinus lepideus showing immune enhancing and immune modulating activity.

Above described Lentinus lepideus comprises the fruit body and mycelium thereof and naturally occurring or artificially cultivated product thereof can be used.

Above extract can be obtained by using distilled water, lower alcohols such as methanol, ethanol and the like, or the mixtures thereof, preferably water.

In accordance with one aspect of the present invention, there is provided an extract of Lentinus lepideus showing immune enhancing and immune modulating activity being prepared by the method comprising the steps of; (a) mixing mycelia of Lentinus lepideus, which are incubated in liquid medium, collected from cultured media using filter, washed with distilled water and dried, or fruit bodies of Lentinus lepideus with distilled water, heating the mixture to extract the active ingredients and centrifuging to obtain the supernatant and (b) concentrating the above supernatant and drying to obtain inventive brown extract comprising heteroglycan and monosaccharides such as glucose, mannose and galactose.

It is another object of the present invention to provide a pharmaceutical composition comprising the above extract as an active ingredient in an amount effective to prevent and treat infectious disease and treat patients suffered from the disorder of immune system together with a pharmaceutically acceptable carrier.

It is another object of the present invention to provide a preparation method of above extract characterized in that: (a) mycelia of Lentinus lepideus stored on agar medium are incubated at the temperature ranging from 15 to 35° C., the speed ranging from 30 to 200 rpm, for the period ranging from 1 to 20 days in liquid medium; (b) And then mycelia are collected from cultured media using filter; (c) the above mycelia are washed with distilled water, dried and mixed with 5 to 20-fold volume of distilled water, lower alcohols such as methanol, ethanol and the like, or the mixtures thereof, preferably hot water at the temperature ranging from 50 to 100° C. for the period ranging from 1 to 48 hours with 2 to 5 times and centrifuged to obtain the supernatant; (d) the above supernatant is concentrated to obtain inventive extract.

And, it is another object of the present invention to provide a use of above extract for the preparation of pharmaceutical composition to prevent infectious diseases and to treat patients suffered from the disorder of immune system.

And, it is another object of the present invention to provide a use of above extract for the preparation of adjuvant of vaccine.

The present invention provides a method for treating a mammal or human afflicted with infectious diseases comprising administering to a mammal or human an effective amount of above extract together with a pharmaceutically acceptable carrier thereof.

It is still another object of the present invention to provide a health food comprising above extract as an active ingredient in an amount effective to enhancing immunity, together with a sitologically acceptable additive.

The pharmaceutical composition for enhancing immunity can contain about 0.01 to 95 w/w %, preferably 0.5 to 80 w/w % of the above extract of present invention based on the total weight of the composition.

An inventive extract may be prepared in accordance with the following preferred embodiment.

For the present invention, above extract can be prepared by following procedure; mycelia of Lentinus lepideus stored on agar medium are incubated at the temperature ranging from 15 to 35° C., preferably from 20 to 30° C., with the speed ranging from 30 to 200 rpm, preferably from 50 to 150 rpm for the period ranging from 1 to 20 days, preferably 5 to 10 days in liquid medium comprising glucose, peptone, yeast extract, molt extract, potassium phosphate, magnesium sulfate and so on. And then mycelia are collected from cultured media using filter. The above mycelia of Lentinus lepideus are washed with distilled water and dried. Dried mycelia of Lentinus lepideus are mixed with 5 to 20-fold, preferably, 10 to 15-fold volume of distilled water, lower alcohols such as methanol, ethanol and the like, or the mixtures thereof, preferably water; treated with hot water at the temperature ranging from 50 to 100° C., preferably from 80 to 100° C., for the period ranging from 1 to 48 hours, preferably 2 to 4 hours with 2 to 5 times and centrifuged to obtain the supernatant. The above supernatant is concentrated with rotary evaporator or under pressure and then dried by freeze-drying or vacuum drying to obtain an extract powder.

And the inventive extract can be prepared by other procedure; dried fruit bodies of Lentinus lepideus are mixed with 5 to 20-fold, preferably, 10 to 15-fold volume of distilled water, lower alcohols such as methanol, ethanol and the like, or the mixtures thereof, preferably water; treated with hot water at the temperature ranging from 50 to 100° C., preferably from 80 to 100° C., for the period ranging from 1 to 48 hours, preferably 2 to 4 hours with 2 to 5 times and centrifuged to obtain the supernatant. The above supernatant is concentrated with rotary evaporator or under pressure and then dried by freeze-drying or vacuum drying to obtain an extract powder.

Therefore, it is another object of the present invention to provide an extract of Lentinus lepideus showing immune enhancing and immune modulating activity being prepared by the steps of: incubating mycelia thereof in liquid medium; collecting from cultured media using filter, washing cultivates with distilled water and drying; mixing the dried mycelia with distilled water, lower alcohols or the mixtures thereof; and treating the suspension with hot water and centrifuging to obtain supernatant; concentrating and then drying to obtain brown powder as said extract.

It is another object of the present invention to provide an extract of Lentinus lepideus showing immune enhancing and immune modulating activity being prepared by the steps of: mixing the dried fruit bodies thereof with distilled water, lower alcohols or the mixtures thereof; and treating the suspension with hot water and centrifuging to obtain supernatant; concentrating and then drying to obtain brown powder as said extract.

It is another object of the present invention to provide an extract of Lentinus lepideus comprising glucose, mannose, galactose and heteroglycan showing immune enhancing and immune modulating activity being prepared by the above preparation method.

In accordance with another aspect of the present invention, there is provided an extract of Lentinus lepideus comprising glucose 55.6%, mannose 18.5%, galactose 25.9% and heteroglycan showing immune enhancing and immune modulating activity being prepared by the above preparation method.

The inventive extract obtained from mycelium and fruit body of Lentinus lepideus through above preparation procedure, are designated as PG101-1 and PG101-2, respectively.

The inventive extracts act on human macrophage/mononuclear cell, regulate cellular transcription factor, NF-κB followed by cytokine activation and increase the levels of various cytokines such as IL-1β, IL-10, IL-12, GM-CSF and IL-18. And it is confirmed that the administration of inventive extract causes the granulocyte/stem cell to differentiate and proliferate in irradiated mice model and has immune-enhancing activity.

In accordance with another aspect of the present invention, there is provided a pharmaceutical composition comprising said extract prepared by above preparation method as an active ingredient for prevention of infectious diseases caused by bacteria or virus and treatment of patients suffered from the disorder of immune system.

Above the disorder of immune system is caused by immune deficiency or suppressed immunity.

The inventive pharmaceutical composition can be treated to the patients suffered from suppressed immunity due to anti-cancer therapy such as chemotherapy and radiotherapy and the patients suffered from AIDS or cancer.

The composition of the present invention has potent immune enhancing and modulating activity and the pharmaceutical composition of the present invention thus may be employed for immune enhancing agent.

And the inventive composition can be administered for the prevention of bacterial infection or viral disease such as influenza and also can be used for adjuvants of vaccine.

In accordance with another aspect of the present invention, there is also provided an immunomodulating agent comprising said extract prepared by above preparation as an active ingredient in an amount effective to treat patients suffered from the disorder of immune system together with a pharmaceutically acceptable carrier.

In accordance with another aspect of the present invention, there is also provided a use of the composition comprising said extract prepared by above preparation method in the manufacture of a medicament for preventing bacterial or viral infection caused by immune suppression or treating the patient requiring immune enhancement.

In accordance with another aspect of the present invention, there is also provided a method of treating a mammal or human afflicted with infectious disease comprising administering to a mammal or human an effective amount of said extract prepared by above preparation method and pharmaceutically acceptable carrier thereof.

It is another of the present invention to provide a method of enhancing immunity in a mammal or human comprising administering to said mammal or human an effective amount of said extract prepared by above preparation method and pharmaceutically acceptable carrier thereof.

It is another of the present invention to provide a treating method comprising administering a pharmaceutical composition comprising said extract prepared by above preparation method to immune-suppressed patients for enhancing immunity.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive extract or compounds of the present invention. The dose may be administered in single or divided into several times per day. In terms of composition, the amount of inventive extract should be present between 0.01 to 95% by weight, preferably 0.5 to 80% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Accordingly, it is another object of the present invention to provide a health food comprising above described extract prepared by above processes and a sitologically acceptable additive to prevent infectious disease and enhancing immunity.

And, it is another object of the present invention to provide a use of above described extract prepared by above processes for the manufacture of the health food employed for enhancing immunity.

It is still another object of the present invention to provide a method for preventing infectious disease, which comprises administering health food comprising above described extract prepared by above processes to enhance immunity.

Above described composition therein can be added to food, additive or beverage. For the purpose of preventing infectious disease and the disorder of immune system, wherein, the amount of above described extract in food or beverage may generally range from about 0.1 to 15 w/w %, preferably 1 to 10 w/w % of total weight of food for the health food composition and 1 to 30 g, preferably 3 to 10 g on the ratio of 100 Ml of the health beverage composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 Ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

In accordance with another aspect of the present invention, there are provided a feed or feed additive essentially comprising said extract prepared by above preparation method as an active ingredient for the livestock, to enhance immunity against infectious diseases.

Inventive feed additive can be added with the range from 0.01 to 95 w/w %, preferably 0.5 to 80 w/w % in feed to enhance immunity against infectious diseases.

In accordance with another aspect of the present invention, there is also provided an use of said extract prepared by above preparation method for manufacture of the livestock feed employed for enhancing immunity against infectious diseases.

In accordance with another aspect of the present invention, there are provided a method of treating the livestock afflicted with infectious disease comprising administering feed and a feed additive containing the above-mentioned extract for enhancing immunity of the livestock.

It will be apparent to those skilled in the art that various modifications and variations can be made in the compositions, use and preparations of the present invention without departing from the spirit or scope of the invention.

### EXAMPLES

The following Examples and Experimental Examples are intended to further illustrate the present invention without limiting its scope.

### Example 1

### Preparation of PG101-1

Mycelium of Lentinus lepideus was distributed by Korea Forestry Research Center. Mycelia of Lentinus lepideus stored on agar medium were incubated at the temperature ranging from 20 to 30° C., with the speed ranging from 50 to 150 rpm for the period ranging from 5 to 10 days in 4 liter of liquid medium (20 g of glucose, 1 g of peptone, 20 g of molt extract, pH 5.5). And then mycelia were collected from cultured media using ADVANTEC filter No. 2 (Toyo, Japan). The above mycelia of Lentinus lepideus were washed with distilled water 5 times and dried at temperature of 56° C. for 16 hours to obtain 14 g of mycelia of Lentinus lepideus.

Dried mycelia of Lentinus lepideus were mixed with 200 Ml of distilled water, heated at 80° C. for 3 hours with 3 times. The extracts were collected and centrifuged at the speed of 5000×g for 10 minutes to obtain the supernatant. Then, the collected supernatant was filtered, concentrated under reduced pressure with rotary evaporator (N-1000, Eyela Co. Japan) and lyophilized to obtain 1.96 g of brown extract powder, PG101-1 (yield: 14%).

### Example 2

### Preparation of PG101-2

11.2 g of dried fruit bodies of Lentinus lepideus obtained from Kangwon National University were cut into small pieces, mixed with 200 Ml of distilled water and heated at 80° C. for 3 hours with 3 times. The extracts were collected and centrifuged at the speed of 5000×g for 10 minutes to obtain the supernatant. Then, the collected supernatant was filtered, concentrated under reduced pressure with rotary evaporator (N-1000, Eyela Co. Japan) and lyophilized to obtain 2.26 g of brown extract powder, PG101-2 (yield: 20.4%).

### Experimental Example 1

### HPLC analysis of PG101-1

PG101-1 and monosaccharide mixture (GalNAC, Xyl, GlcNAc, Glc, Man, Fuc, Gal) were properly diluted with distilled water and 50 μl of diluents were concentrated and dried in the GlycoTAG vial. Samples were hydrolyzed with 4N HCl/4M trifluoroacetic acid at 100° C. for 4 hr, and 100 pmol of pyridyl amino acid (PA) was then added thereto. PA derivatives were analyzed using HPLC system (1100 series, Agilent Co., U.S.A.) with PALPAK Type A column. The amount of each monosaccharide was calculated by a peak height method (flow rate, 0.3 Ml/min, eluant; 0.7M Borate-K (pH 9.0): acetonitrile=9:1, detector Ex 310 nm, column temperature 65° C.).

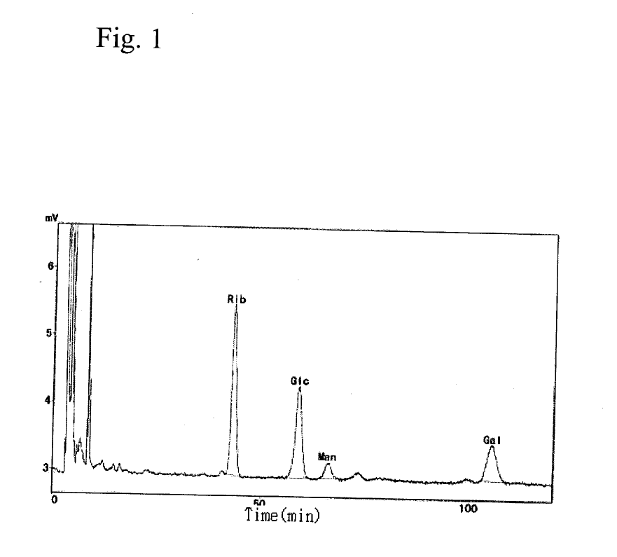

As the result, it was confirmed that PG101-1 comprised monosaccharide part, i.e., glucose 55.6%, mannose 18.5%, galactose 25.9% and heteroglycan (FIG. 1).

### Experimental Example 2

### Limulus Test

To detect the endotoxin in PG 101 prepared in Example, PG 101 was assayed under endotoxin-free experimental condition using a Limulus Amebocytes Lysate (LAL) Pyrogen Kit (Biowhittaker, Walkerswille, Md.). Experiments were performed according to the manufacturer's protocol. Briefly, 100 μl of standard reagent, PG101-1 or controls were mixed with 100 μl of LAL reagent and incubated for 1 hour at 37° C. and then the gelatinized status of each tube was then examined.

It is confirmed that the amount of endotoxin in PG101-1 was less than 0.015 EU/mg.

### Experimental Example 3

### The Effect of PG101-1 on the Production of Cytokines in Mononuclear Cell

To determine the effect of PG101-1 as immune enhancer, PG101-1 was treated to the human peripheral blood mononuclear cell (PBMC) and the changes of cytokines were measured.

Blood samples were obtained from healthy volunteers and treated with EDTA as an anticoagulant. PBMCs were isolated by Ficoll-Hypaque (Amersham Pharmacia Biotech AB) gradient centrifugation. Approximately 1×10⁷ cells were incubated with DC11b/Mac-1 or CD19 (BD PharMingen, CA, USA) antibody at 0° C. for 40 min. Cells were placed on the dishes coated with anti-mouse IgG (10 μg/Ml) for 1 hr at 4° C. Cells were then washed with PBS containing 1% FBS, and attached cells were collected by scrapper. For isolation of T cells, PBMCs were incubated with microbead conjugated with mouse monoclonal antibody to human CD3 (Miltenyi Biotech, Heidelberg, Germany) for 30 mins at 4° C. Following above incubation, the mixture was passed through a magnetics column according to the manufacturer's instruction. The column was washed with PBS containing 1% albumin, and bound cells were eluted with the same buffer. Isolated cells were subjected analyzing by FACS, and the cells showing over 80% purity for a given antibody, were used for this experiments. Cells were incubated with 10 μg/Ml of PG101-1 at 37° C. under an atmosphere containing 5% CO₂. As a positive control, 10 ng/Ml lipopolysaccharides (LPS, Sigma, St Louise Mo.)) was treated to the cells. ELISA kits for cytokines in cell culture medium were purchased from Endogen (Woburn, Mass.). The each optical density was measured by microplate reader (Versamax tunable microplate reader, Molecular Device, USA) and calibrated according to the standard conc. curve. Tested cytokines could be classified into four categories, depending on the magnitude of effects of PG101-1 on these cytokines.

Background levels of many cytokines were either undetectable or low, but PG101-1 treatment group shows dramatically increased levels of a small number of cytokines. The fist class of cytokines (Class I) comprising TNF-α and IL-1β were increased by more than three times of magnitudes by PG101-1, and the effect of PG101-l was significantly higher than that of LPS as positive control at a given concentration. The second class of cytokines (Class II) comprising IL-10 and IL-12 whose levels were increased by on average two times of magnitude and the third class of cytokines (Class III) comprising GM-CSF and IL-18 whose levels were increased to the extent that less than 10 fold upon PG101-1 treatment. This group was characterized by its significantly high background levels of cytokines prior to PG101-1 treatment. Lastly, the fourth class of cytokines (Class IV) comprising IFN-γ and IL-4, did not respond to PG101-1 even at high concentrations.

Finally, as shown in Table 1, it was confirmed that PG101-1 could increase the levels of several selective cytokines and 10 μg/Me of PG101-1 could lead the maximum effect in the experiment using by most cytokines.

TABLE 1

Fold Induction

| Class | Cytokines | PG101-1 (10 μg/Ml) | LPS(10 ng/Ml) |
|---|---|---|---|
| Class I | TNF-α | 1,400 ± 21 | 915 ± 61 |
| | IL-1β | 1,540 ± 391 | 97 ± 2 |
| Class II | IL-10 | 223 ± 17 | 201 ± 78 |
| | IL-12 | 223 ± 24 | 136 ± 2 |
| Class III | GM-CSF | 9 ± 0.4 | 7 ± 0.3 |
| | IL-18 | 8 ± 0.6 | 4 ± 0.6 |
| Class IV | IFN-γ | — | — |
| | IL-4 | — | — |

### Experimental Example 4

### The Effect of PG101-1 Concentration on Cytokine

To determine the effect of PG101-1 having various concentrations on cytokine production, the experiment was executed according to the procedure in above Experimental Example 3.

After treatment of 0.1, 1, 10 and 100 μg/Ml of PG101-1, PBMCs were incubated in the condition of 37° C., 5% CO₂ for 24 hours. And then the levels of class I, II and class III cytokines were measured by commercial ELISA microplate reader.

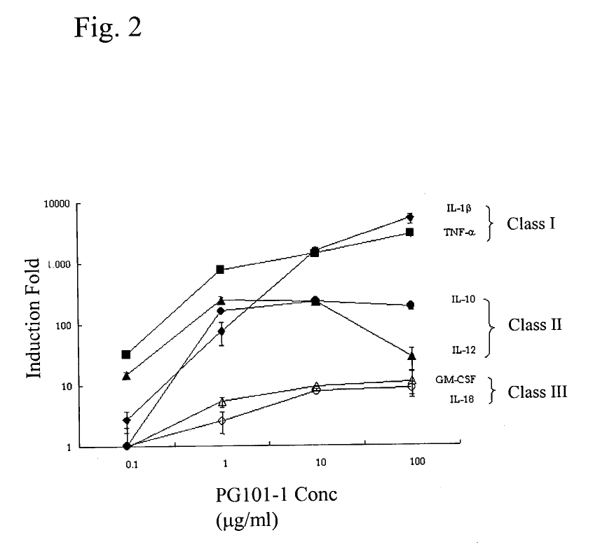

For class I and class II cytokines, their levels were increased in a logarithmic manner up to 1 μg/Ml PG101-1. At higher concentrations, the effects of PG101-1 became less pronounced. Levels of class I cytokines reached at most high peak at the concentration of 100 μg/Ml PG101-1, while the highest levels of Class II proteins were achieved at the concentration of 10 μg/Ml PG101-1. The response of Class III molecules was characteristically slow and was reached at its highest level at the concentration of 10 μg/Me. Most of cytokines responded to PG101-1 in a dose-dependent manner (FIG. 2).

### Experimental Example 5

### The Effect of PG101-1 on Cytokine

To determine the effect of PG101-1 on cytokine production with passage of time, the experimental procedure was performed according to the above Experimental Example 3.

After treatment of 10 μg/Me of PG101-1, PBMCs were incubated in the condition of 37° C., 5% CO₂ and then the levels of class I, II and class III cytokines were measured at 2, 6, 12 and 24 hours using by commercial ELISA microplate reader.

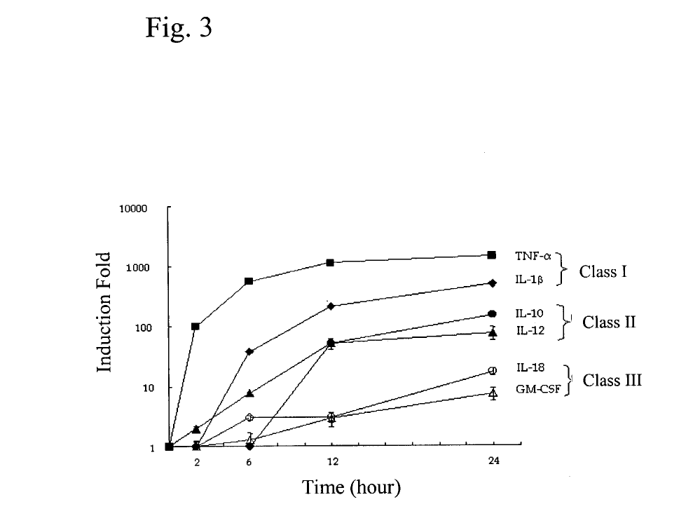

Effects of PG101-1(at 10 μg/Ml) showed also time-dependent and reached at its highest peak at 12 to 24 hrs after treatment with most cases (Table. 2 and FIG. 3). At the given detection system, TNF-α was the first detectable cytokine. At the moment of 2 hours after treatment of PBMCs with 10 μg/Ml of PG101-1, the level of TNF-α was increased by 100 fold. The response of IL-1β was slower than that of TNF-α. Its level was maintained during the first 2 hours, but increased by approximately 40 fold at 6 hour post-treatment. The 50-fold induction of PG101-1 on Class II cytokines was found at 12 hours after treatment. It is interesting to note that response of IL-12 was detectable as early as 2 hours, but only at a level of 2-fold. The 10-fold activation of Class III cytokines, GM-CSF and IL-18, was found at 24 hours after PG101-1 treatment. In summary, when 10 fold activation was used as a criterion, the sequence of activation of cytokines was [TNF-α→IL-1β→(IL-10, IL-12)→(GM-CSF, IL-18)].

TABLE 2

| | | Time (hour) | | | |
|---|---|---|---|---|---|
| Class | Cytokine | 2 | 6 | 12 | 24 |
| I | TNF-α | 100 ± 14 | 550 ± 26 | 1105 ± 8 | 1450 ± 34 |
| | IL-1β | 1 ± 0.4 | 38 ± 2 | 211 ± 5 | 493 ± 40 |
| II | IL-10 | 1 ± 0.2 | 1 ± 0.5 | 50 ± 1 | 150 ± 30 |
| | IL-12 | 2 ± 0.3 | 7.7 ± 0.4 | 51 ± 22 | 75 ± 39 |
| III | GM-CSF | 1 ± 0.5 | 1.3 ± 0.7 | 2.9 ± 1.6 | 7.5 ± 3.6 |
| | IL-18 | 1 ± 0 | 3 ± 0.5 | 3 ± 1.2 | 17 ± 5 |

### Experimental Example 6

### Major Target Cell Type for PG101-1

To determine the cell type affected by PG101-1, PBMCs were sorted to macrophage, T cells and B cells by a panning method using antibodies specific to each cell type. PBMCs were incubated with antibodies to cell surface proteins, CD11b/Mac-1 (for monocyte/macrophage), and CD19 (for B cells). Cells were then placed on a culture dish coated with anti-IgG antibody for isolation. After washing, bound cells were collected by scrapping. For T cells, magnetic bead conjugated with CD3 antibody was used, and bound cells were isolated by Mini-MACS. The purities of isolated cells were determined by FACS using the same antibodies. Each cell population showed over 80% purity for respective production.

And to verify the cytokine production affected by PG101-1 in each cell type, the experiment was performed according to the above Experimental Example 3.

As shown in Table 3, neither B cells nor T cells responded to PG101-1 in terms of cytokine production. On the contrary, CD11b/Mac-1 positive cells, which are thought to contain a predominantly monocyte/macrophage lineage, responded to PG101-1 in a manner with that similar to PBMCs, suggesting that macrophage might be the major cell target of this fungal extract PG101-1.

TABLE 3

| | | Cell Type | | | |
|---|---|---|---|---|---|
| | | | Macrophage | B cell | T cell |
| Class | Cytokines | PBMCs | (CD11b+) | (CD19+) | (CD3+) |
| I | TNF-α | +++¹⁾ | +++ | − | − |
| | IL-1β | +++ | ++ | − | − |
| II | IL-10 | ++²⁾ | − | − | − |
| | IL-12 | ++ | + | +/− | − |
| III | GM-CSF | +³⁾ | +++ | − | − |
| | IL-18 | + | − | − | − |
| IV | IFN-γ | −⁴⁾ | − | − | − |
| | IL-4 | − | − | − | − |

¹⁾++++: Denotes above 500 times, ²⁾++: denotes 50-500 times, ³⁾+: denotes 10 times ⁴⁾−: denotes no change

### Experimental Example 7

### The Determination of the Amount of Cytokines in Human Cell Line Treated with PG101-1

For further confirmation subsequent Experimental Example 6, human cell lines of various origins were used. Human erythroblastic leukemia cell line K562, human T cell line Jurkat cell, human embryonic kidney cells 293, human promonocytic cell line U937 were obtained from American Type Culture Collection (ATCC, Rockwille, Md.).

Each cell line was maintained with DMEM or RPMI 1640 medium supplemented with 200 μg/Ml streptomycin and 120 μg/Ml penicillin G (Sigma, St Louise Mo.) containing 10% FBS (GIBCO BRL).

Cell lines were incubated with 250 μg/Ml PG101-1 at 37° C. under an atmosphere containing 5% CO₂ for 24 hours. Levels of cytokines, i.e., TNF-α, INF-γ, IL-1β, IL-4, IL-10, IL-12, GM-CSF and IL-18 were measured using commercially available ELISA kits according to the manufacturer's instruction. ELISA kits for the first 7 cytokines were purchased from Endogen (Woburn, Mass.), while the level of IL-18 was measured using the kit from Medical and Biological Laboratories (Nagoya, Japan).

In the result, Jurkat and K562 lines were hardly affected, if at all, by PG101-1. On the contrary, the response of U937 to PG101-1 was similar to that of PBMC or primary monocyte/macrophages (Table 4). These data further confirmed that cells of a macrophage lineage are the major targets of PG101-1.

TABLE 4

| | | Cell line | | |
|---|---|---|---|---|
| Class | Cytokine | U937 | K562 | Jurkat |
| I | TNF-α | ++²⁾ | −⁴⁾ | +³⁾ |
| | IL-β | +++¹⁾ | − | − |
| II | IL-10 | ++ | − | − |
| | IL-12 | ++ | − | − |
| III | GM-CSF | ++ | ++ | − |

¹⁾++++: Denotes above 500 times,
²⁾++: denotes 50-500 times,
³⁾+: denotes 10 times
⁴⁾−: denotes no change

### Experimental Example 8

### NF-κB Activation by PG101-1

8-1. Cellular Transient Transfection Using Reporter Plasmid.

In order to understand the molecular mechanism underlying activation of various cytokine by PG101-1, we first tested the effects of PG101-1 on cellular transcription factors and confirmed DNA transfection in cells by the procedure described in the literature (Pear et al; P. N. A. S., USA, 90, pp8392-8396, 1993).

Reporter plasmids containing binding site for NF-κB, AP-1 (Activator Protein-1), and CRE (cyclic AMP response element) were purchased from STRATAGENE (CA, USA). These plasmids contain the luciferase coding sequence as a reporter gene. Activation of transcription factors was assayed by transiently transfecting 293 cells with 2 μg reporter plasmid, together with 0.5 μg β-galactosidase plasmid using the calcium phosphates method (Pear et al., P.N.A.S. USA, 90, pp8392-8396, 1993). Six hours later the transfection, cells were treated with appropriate concentration of PG101-1 for 18 to 24 hr. Cell lysates were prepared and assayed for their luciferase activity using the Luciferase Reporter kit (Promega) and Reporter Microplate Luminometer (Turner Instruments, Sunnyvale, Calif.).

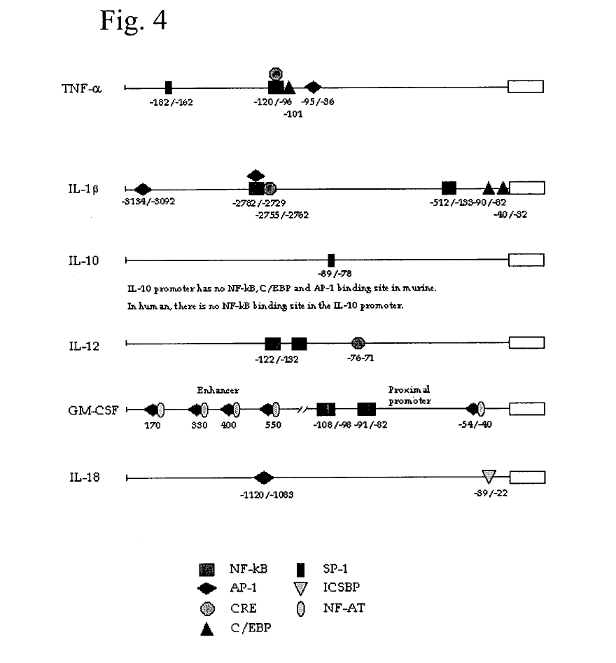

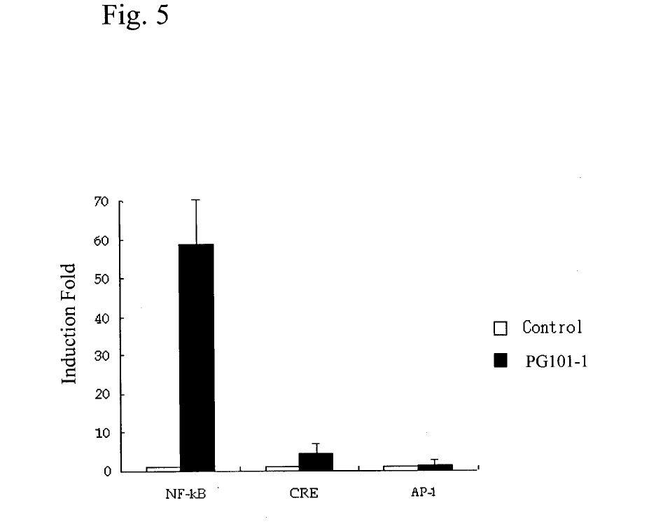

Structures of promoters of cytokine genes are shown in FIG. 4. NF-κB binding sequences are the most frequently found element, present in four promoters except for those of IL-10 and IL-18 (Hiscott et al., Mol. Cell. Biol., 13, pp6231-6240, 1993). AP-1 binding sites are also present in promoters of TNF-α, IL-1β, and IL-12 containing the nucleotide sequences that can interact with CREB (Chandra et al., J. Immunol., 155, pp4535-4543, 1995; Yao et al., J. Biol. Chem., 272, pp17795-17801, 1997). Based on this observation, the effects of PG101-1 on three transcription factors were determined by transient transfection assays involving reporter plasmid at first. In these plasmids, a synthetic promoter drives gene expression that consists of minimum general transcription machinery and multiple copies of short nucleotide sequences to which respective transcription factors bind thereto. The coding sequence for luciferase is located at downstream position from this control region and its expression is dependent upon the presence of respective transcription factors. 293 cells were transfected with respective reporter plasmids and treated with 250 μg/Ml of PG101-1. 24 hours later, cell lysates were prepared and used to measure the level of luciferase activity. Treatment with PG101-1 showed increased the level of luciferase activity by 60-fold when the NF-κB reporter plasmid used (FIG. 5). The level of luciferase activity from a CREB reporter plasmid was also increased, but by less than 5-fold. It is confirmed that PG101-1 did not have any significant effect on AP-1 (FIG. 5).

8-2. Electro Mobility Shift Assay (EMSA).

For NF-κB, U937 cells were cultured at 5×10⁵/Ml for 16-20 hours and stimulated with PG101-1 as indicated time. Nuclear extracts were prepared using by the treated or not treated cells with PG101-1 (Phares et al., J. Virol., 66, pp7490-7498, 1992). The oligonucleotide probe corresponding with −86, −116 region of HIV LTR promoter known to be inter-reacted with NF- κB, was reacted together with labeled ³²P. The reaction mixture containing 3 μg of poly(dI-dC), 0.3 ng of double stranded [³²P]-labeled oligonucleotide NF-κB probe (5′-ATCCTCCGCTGGGGACTTTCCAGGGAGGA-3′) and 3 μg of nuclear extracts in binding buffer (10 mM Tris-HCl, pH 7.5, 50 mM NaCl, 1 mM DTT, 5% glycerol, 2 μg of bovine serum albumin, 3 mM GTP) were prepared to the extent that its final volume was made to 20 μg. After incubation for 20 min at room temperature, the reaction solution was analyzed on a low-ionic strength 4% polyacrylamide gel using by running buffer (6.7 mM Tris-HCl, pH 7.5, 3.3 mM sodium acetate, pH 7.0, 1 mM EDTA, pH 7.5). The specificity of the retarded complex was confirmed by competition test with 40-fold excess of cold wild type or mutant oligonucleotide (5′-GATCCTCCGCTCTCGACTTTCCAGGGAGGA-3′).

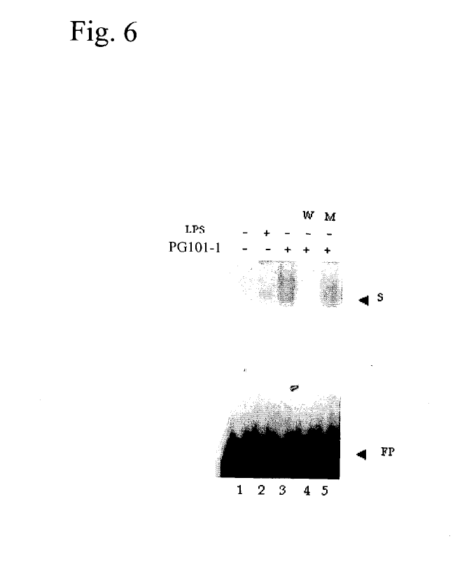

At the result, untreated cells showed a low basal level of NF-κB activity, however, treatment cells with PG101-1 showed significantly increased amount of electrophoretically retarded DNA-protein complex (FIG. 6, lane 3).

This complex was specific to NF-κB because they were effectively competed by the unlabelled wild type NF-κB sequence but not by a mutant oligonucleotide, which was identical to the wild type probe except for three nucleotide changes (FIG. 6, lane 4 & 5).

8-3. The Effect of PG101-1 & PDTC (Pyrrolidine Dithiocarbamate) on NF-κB.

For further confirmation on the involvement of NF-κB in PG101-1 mediated activation of cytokines, it was also tested whether cytokine activation by PG101-1 could be suppressed by PDTC (pyrrolidinedithiocarbamate, Sigma, St Louise Mo.) which specifically inhibits the activity of NF-κB (Ziegler-Heitbrock et al., J. immunology, 151, pp6986-6993, 1993).

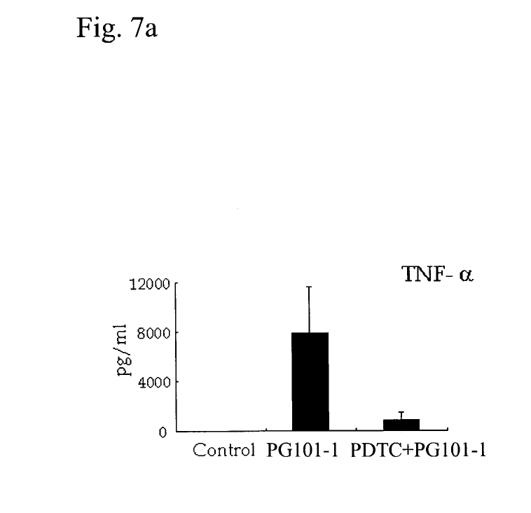

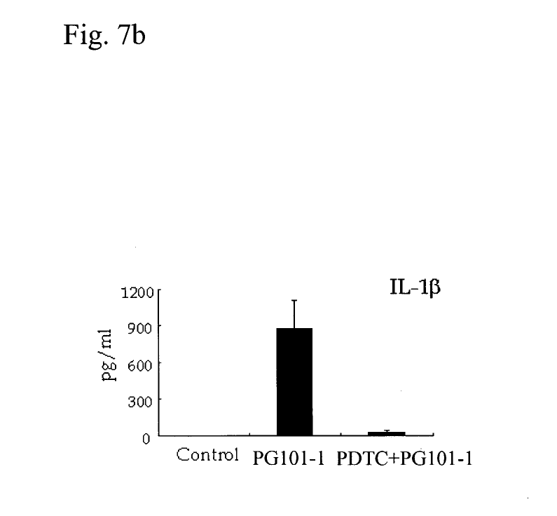

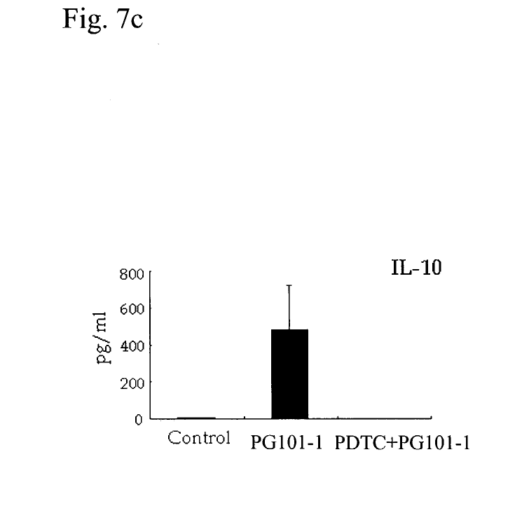

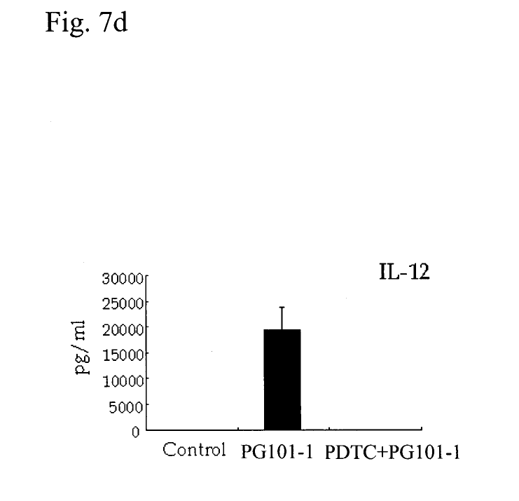

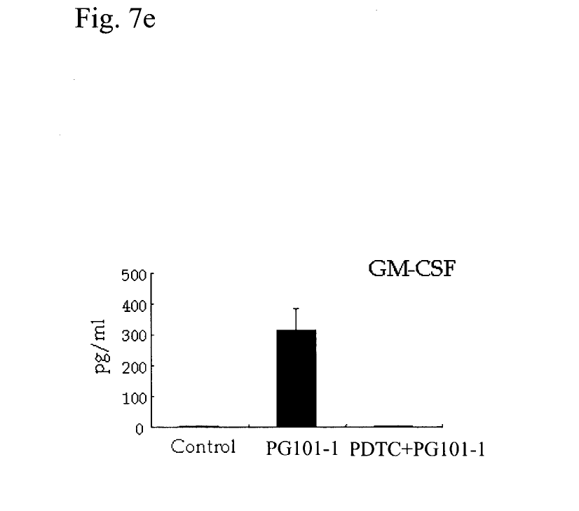

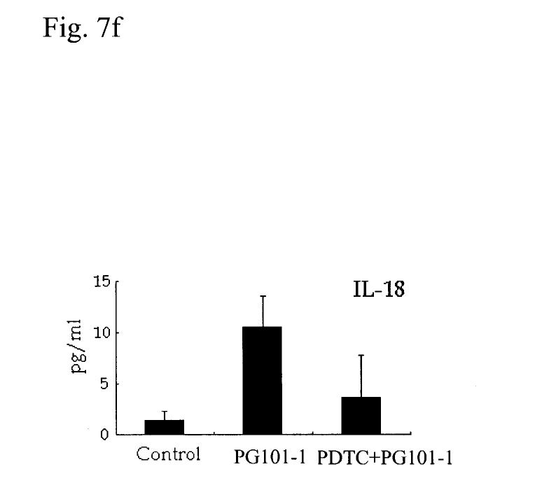

The levels of each TNF-α, IL-1β, IL-10, IL-12 and GM-CSF were completely suppressed by PDTC at 100 μM concentration (FIGS. 7a, 7b, 7d & 7e), while the expression of IL-18 whose promoter does not contain the NF-κB binding site, was not inhibited by PDTC (FIGS. 7c & 7f).

It is interesting to note that activation of IL-10 was also inhibited by PDTC, although its promoter did not contain the NF-κB sites. It is confirmed that PG101-1 controls IL-10 by using NF-κB indirectly. It is confirmed that these cumulative data clearly indicated that PG101-1 regulates the expression of affected cytokines mainly by controlling cellular transcription factor NF-κB.

### Experimental Example 9

### Toxicity of PG101-1

To examine the toxicity of PG101-1, MTT assay was performed on various human cells.

U937 (leukemia cell line), HT1080 (cancer cell line), and PBMCs were placed at 5000 cells per well in a 96-well plate. Cells were grown in the presence of PG101-1 at the concentration range from 5 to 5000 μg/Ml at 37° C. in a 5% CO₂ incubator. After 96 hours of incubation with PG101-1, viable cells were stained with MTT (5 mg/Ml) for 30 min. The medium was then removed and the produced formazan crystals were dissolved by the addition of 200 μl dimethylsulfoxide (DMSO). Absorbance was measured at 540 nm using an ELISA microplate reader. The IC₅₀ value was defined as the drug concentration that resulted in a 50% reduction in cell number compared with untreated controls. These values were derived from semi-log plots of percent viability (%; absorbance of treated sample/absorbance of untreated control×100) versus drug concentration.

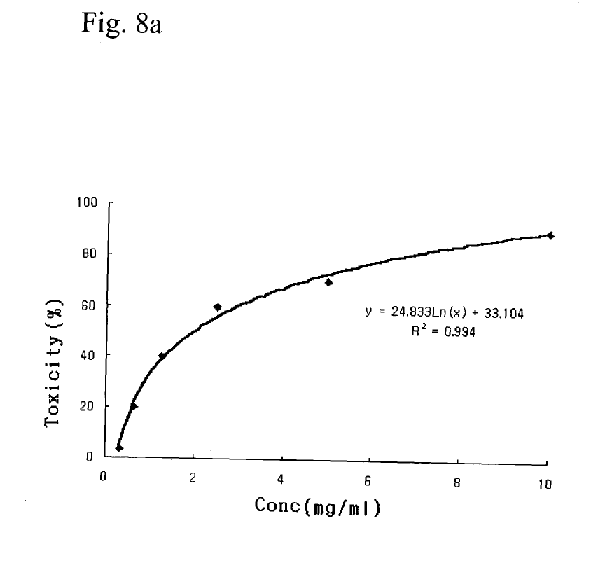

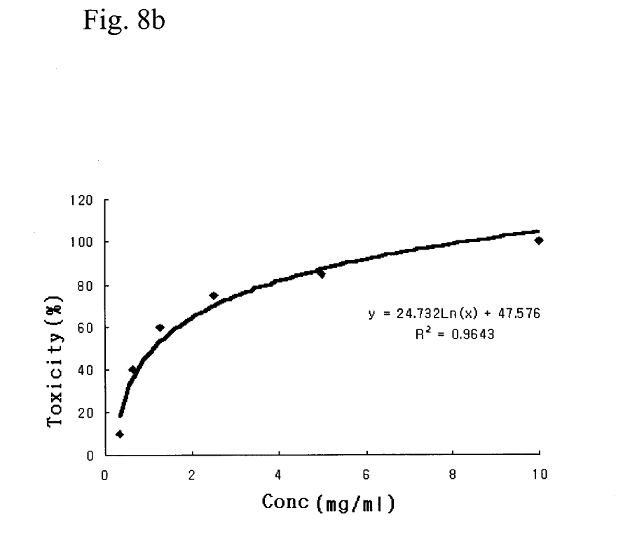

As shown in FIGS. 8a and 8b, the level of PG101-l showing 50% growth inhibition (IC₅₀) after 96 hours of incubation was 976 μg/Ml for HT1080, 1951 g/Ml for U937 and 703 μg/Ml for PBMCs.

### Experimental Example 10

### Colony Forming Analysis

To determine the immuno enhancing activity of PG101-1 through colony forming formation experiment after the irradiation of radioactive light.

Female BALB/c mice, 8-10 weeks of age and weighing 18-20 g, were purchased from the Seoul National University Laboratory Animal Center.

Mice were first divided into the four groups. Each group consisted of 9 mice and was placed in ventilated containers and exposed bilaterally to gamma irradiation from a cobalt-60 source teletherapy unit (Model V9, Picker). Exposure time was adjusted in order that the average amount of absorbed irradiation in each animal tissue was 6 Gy.

The first group mice were not irradiated but treated with water and the second group mice were irradiated with 6 Gy and then treated with PBS (phosphate buffered saline, pH 3.2). The third group mice were also irradiated and then treated with 10 mg of PG101-1 prepared in Example 1 per animal orally every day for 24 days. The fourth group mice were also irradiated and then injected subcutaneously with 3 μg G-CSF (granulocyte-colony stimulating factor) on a daily basis to induce bone marrow cell to differentiate to granular lymphocyte. At 8, 16 and 24 days after the treatment, the first, second and third group mice were sacrificed by cervical dislocation and the bone marrow cells from intact mice were drawn by flushing femoral bones with an IMDM medium and the femoral bone marrow cells were placed on plate and incubated in triplicate at a concentration of 1×10⁵ cells/Ml in semisolid methylcellulose supplemented with 1% methylcellulose in IMDM, 15% FBS, 10⁴ M 2-mercaptoethanol, 10 ng/Ml IL-3, 50 ng/Ml SCF, 3 units/Ml erythropoietin (Stem Cell Technologies, Vancouver, Canada) as a source of colony stimulating activity at 37° C. in CO₂ incubator a humidified atmosphere containing 5% CO₂. The numbers of colonies were counted under the microscope after 10 days of culture. Additionally, the bone marrow cells of non-irradiated fourth group mice was collected from the drawn by flushing femoral bones as a control group and incubated in IMDM medium and the numbers of colonies formed was determined in the procedure identical with above procedure. Cells in which number was over 50 were counted as colonies, and the average number of colonies and standard errors were calculated from triplicated wells.

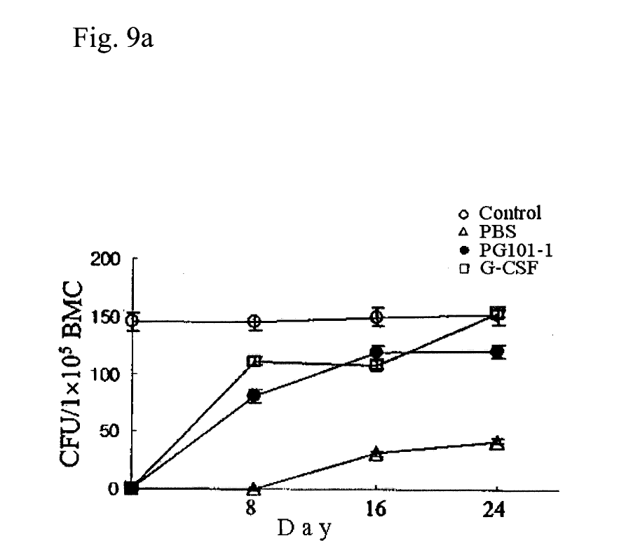

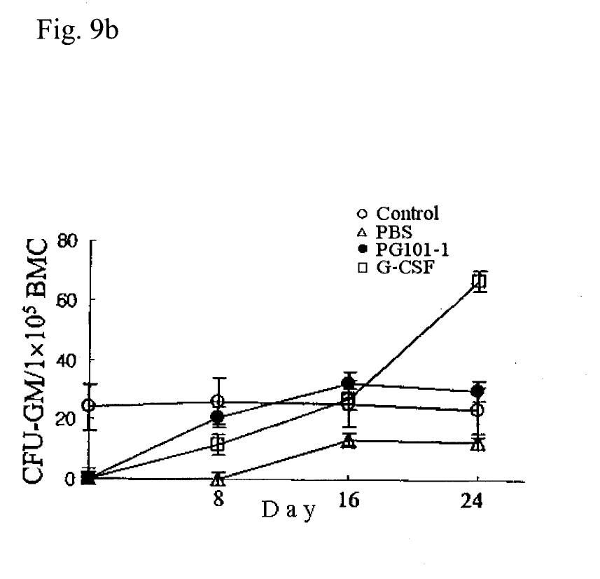

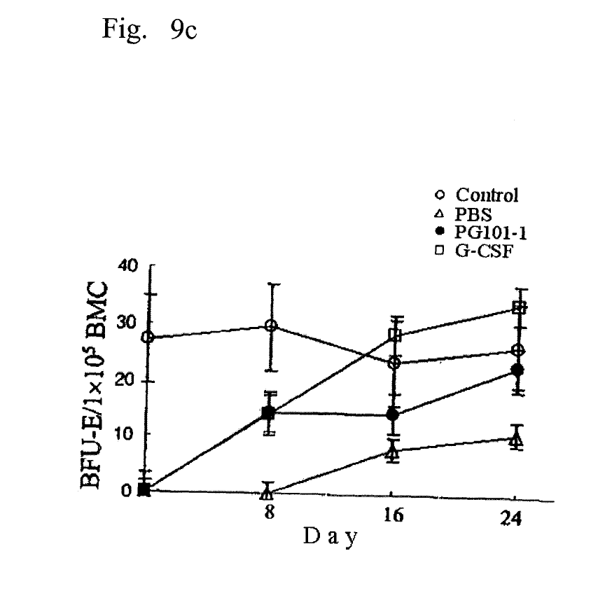

At the results, in the control mice fed with PBS, it showed almost no CFC formed at day 8, and remained very low until day 24. On the contrary, in bone marrow cells from PG101-1-treated mice, the number of CFC was already close to the normal level at day 8, and continued to show high numbers during the 24-day period. G-CSF treated mice also produced the normal number of CFC (FIG. 9a).

In PBS-treated control mice, neither CFU-GM (colony forming unit-granulocyte and monocyte-commited stem cells) nor BFU-E (Burst forming unit-erythroid) was formed properly and their number remained significantly lower than that in naive mice. On the contrary, the number of CFU-GM in PG101-1 treated mice was comparable to that in non-irradiated normal mice at all time points (FIG. 9b). Consistent with other investigators' findings, repeated injections of G-CSF resulted in an abnormal increase of CFU-GM. The number of BFU-E in PG101-1 treated mice at day 8 was approximately half of that in naive mice, but it reached at almost the normal level at day 24(FIG. 9c).

These results suggested that the treatment of PG101-1 could induce the formation of various early staged progenitor cells, including those of granulocytes and macrophages.

### Experimental Example 11

### Flow Cytometric Analysis

In order to confirm the therapeutic effect of the PG101-1 on bone marrow cell and cell types, the experiment was performed as following flow cytometric analysis.

For the antibody in the experiment, ER-MP20 (anti-Ly-6C, PharMingen Co.) conjugated with ER-MP12 (antiCD31, PharMingen Co.) and FITC, Boitinized c-kit (anti-CD117, PharMingen Co.), Gr-1 (anti-Ly-6G, PharMingen Co.) and Streptavidin (Sav-PE, Sigma Co.) conjugated with phycoerythrin (PE, secondary antibody) were used.

11-1. Analysis of Progenitor Cells Using ER-MP12 and ER-MP-20 Antibody

The femoral bone marrow cells were washed with phosphate-buffered saline (PBS) containing 2% fetal bovine serum and 0.1% sodium azide (FACS buffer) and preincubated in the same buffer with 25% normal rat serum for 30 min at 4° C. Cells were reacted with 100 μl aliquots of cocktail containing saturating amounts of Biotinylated anti-mouse ER-MP12 for 30 min at 4° C. in a dark place. Cells were then washed twice with FACS buffer and reacted with PE-conjugated streptavidin or FITC-conjugated anti-mouse ER-MP20 for 30 min at 4° C. Cells were washed again and suspended in 500 μl FACS buffer. For the detection of c-Kit and Gr-1 in bone marrow cells, PE-conjugated anti-mouse c-Kit antibody or PE-conjugated anti-mouse Gr-1 antibody was respectively used under identical experimental conditions (30 min, 4° C.). Flow cytometry was performed on a FACSort (Becton Dickinson, San Jose, Calif.) with CellQuest (Becton Dickinson) data acquisition and analysis software. In all cases, nonspecific staining was controlled by isotype matched antibodies.

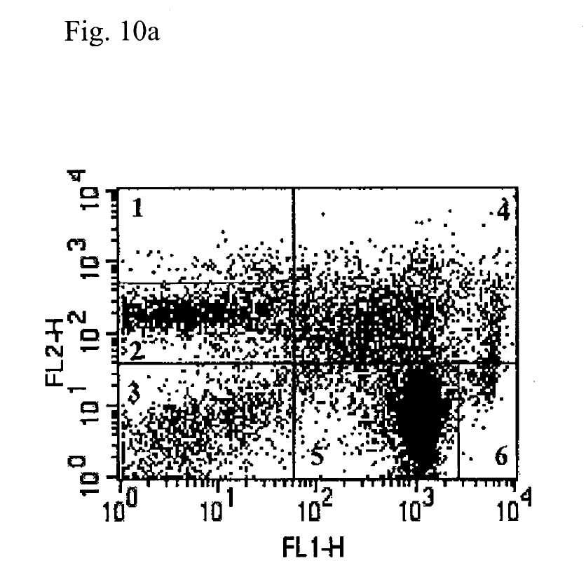

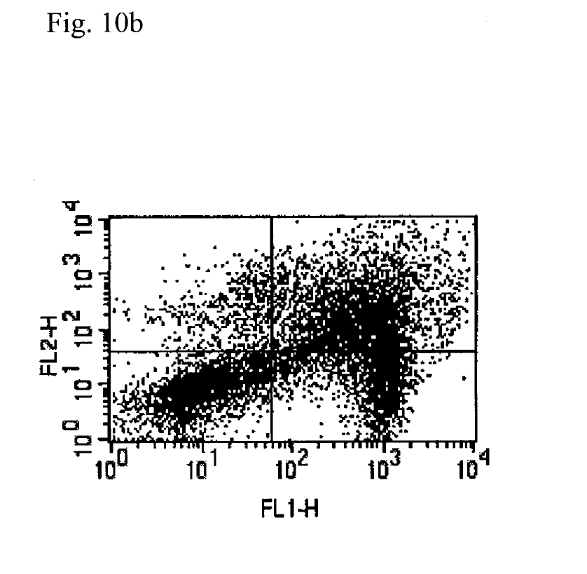

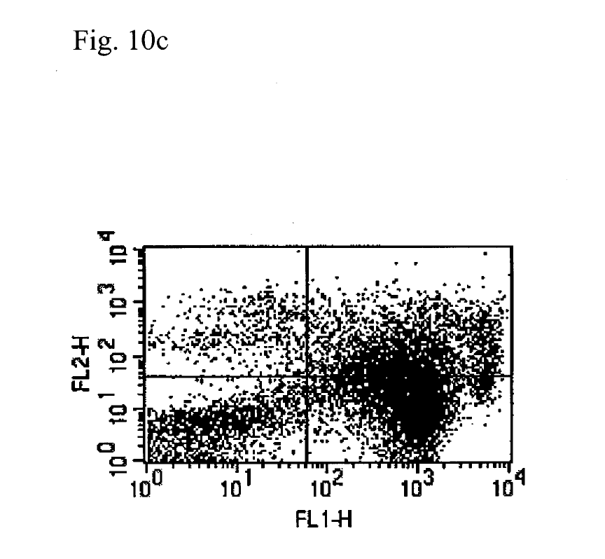

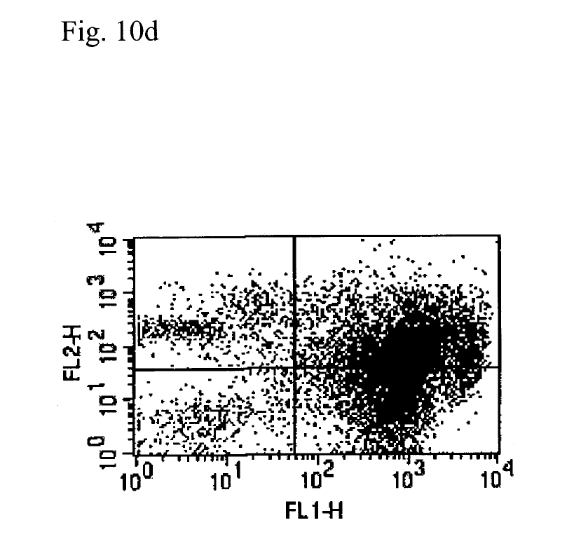

ER-MP12 interacts with CD31 and PECAM-1 on the cell surface which surface, which are expressed at the early stage of hematopoiesis in the progenitor cells, but absent during the later stage of differentiation (Slieker W A et al.; Int. Immunol., 5, pp1099-1107, 1993). ER-MP20 binds to the Ly-6C protein that is expressed during the differentiation of the colony forming cell to granulocytes or monocytes. The relative presence of these proteins has been used to differentiate 6 different subpopulations in the bone marrow as shown in FIG. 10a. For convenience, each subpopulation is named from subsets 1 to 6. By a two-color flow cytometric analysis, changes in the bone marrow subpopulations were analyzed following treatment with PG101-1. As controls, bone marrow cells from irradiated mice treated with PBS or G-CSF were also used. The typical double staining pattern of bone marrow cells in the normal mouse is shown in FIG. 10a. When irradiated mice were treated with PBS, the number of cells in subsets 3 is significantly increased (FIG. 10b). These subsets are thought to consist mainly of erythroid cells and unknown damaged population. The seemingly increased number of cells in subset 3 probably resulted from the increased number of damaged cells. When mice were administered with PG101-1, the number of cells in subsets 4 and 5 (ER-MP12⁺20⁺ and ER-MP12⁻20^{med}) were noticeably increased and became comparable to that in normal mice (FIG. 10c). The similar observation was made in G-CSF treated animals (FIG. 10d). These results suggested that PG101-1 might target granulocytes and/or early myeloid progenitor cells.

11-2. Analysis of Progenitor Cells Using c-Kit and Gr-1

In order to confirm the therapeutic effect of the PG101-1 on the differentiation of bone marrow, a sort of granular lymphocyte, the experiment was performed in identical procedure described in Experiment 11-2 as follows.

To investigate the expression of c-Kit and Gr-1 in bone marrow cell, anti-mouse c-Kit antibody conjugated with PE or anti-mouse Gr-1 antibody conjugated with PE were used under identical experimental condition (4° C., 30 min). For analysis of progenitor cell, FACSort (Becton Dickinson Co.) and Cellquest (Becton Dickinson Co.) software for assessing the data and analysis, were used and isotype antibody was used to determine nonspecific staining in all experiments.

c-Kit is the receptor to the stem cell factor. When stem cells are differentiated to the progenitor cells of the next stages, the level of the c-Kit protein is one of the first few changes that occur, shifting from low to high (Doi H et al., P.N.A.S. USA, 94, pp2513-2517, 1997). However, when cells mature further to lineage-committed cells, c-Kit expression is known to decrease (Muller-Sieburg C E, J. Exp. Med., 167, pp1825-1840, 1988).

We also examined the Gr-1 protein, which is absent from primitive progenitor cells, but is highly expressed when progenitor cells are committed to the macrophage or granulocyte lineage (Fleming T. J. et al., J. Immunol., 151, pp2399-2408, 1993). Cells were analyzed by a single-color flow cytometric analysis using antibodies to c-Kit or Gr-1.

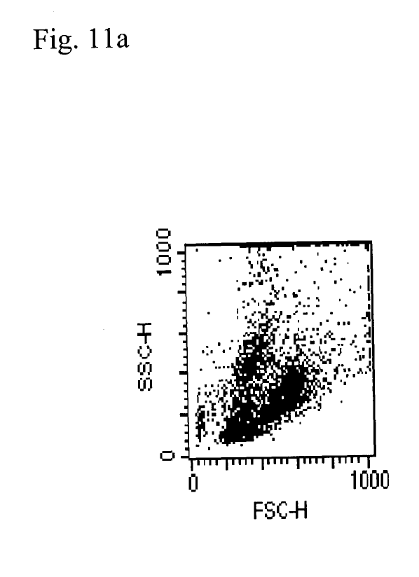

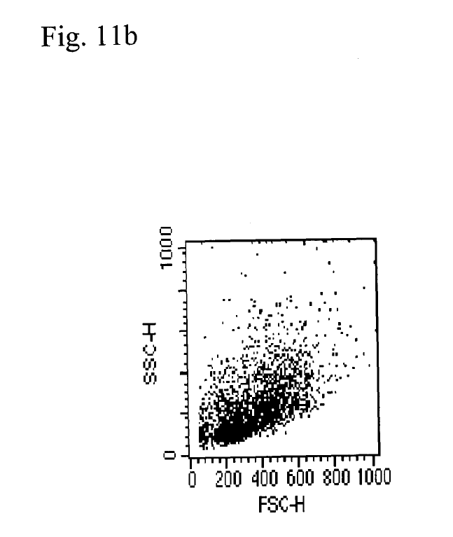

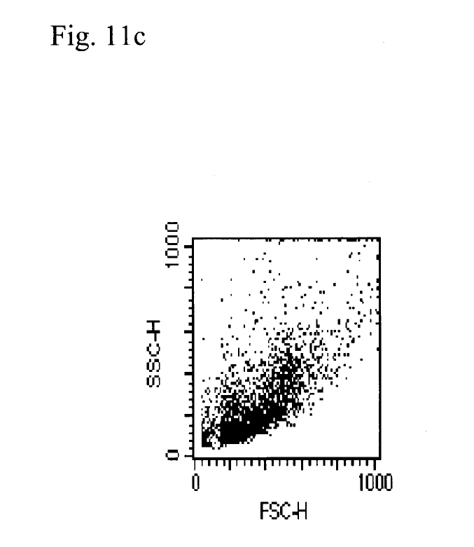

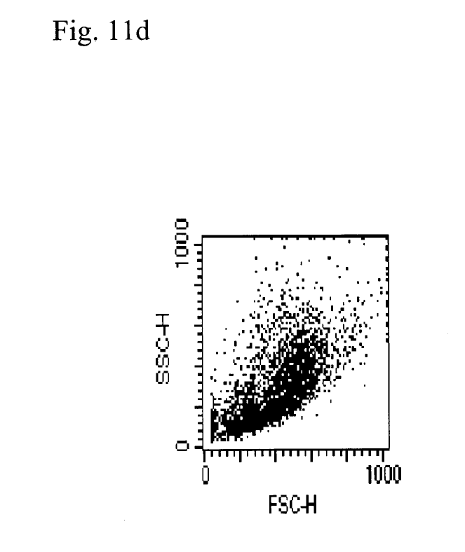

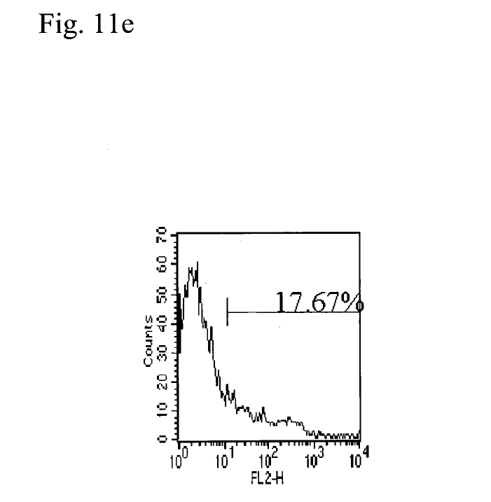

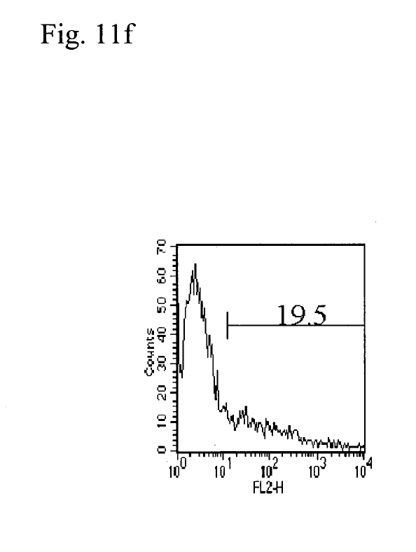

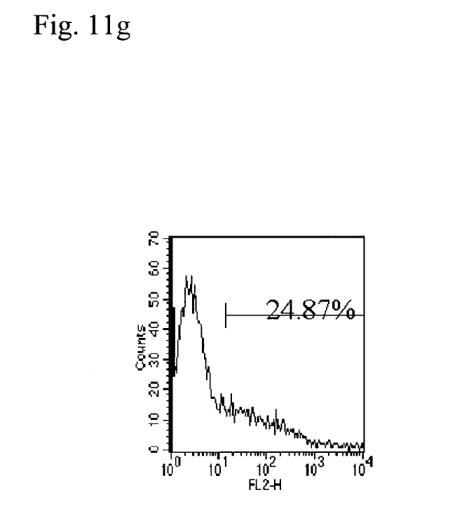

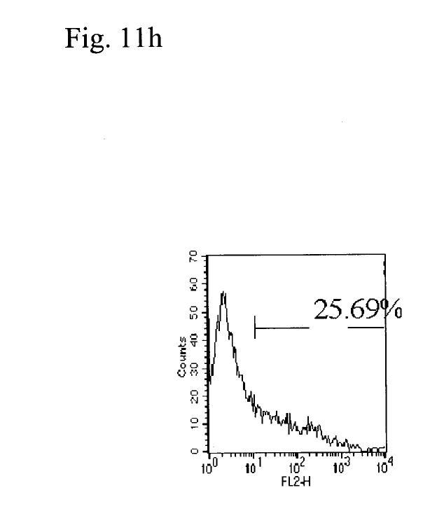

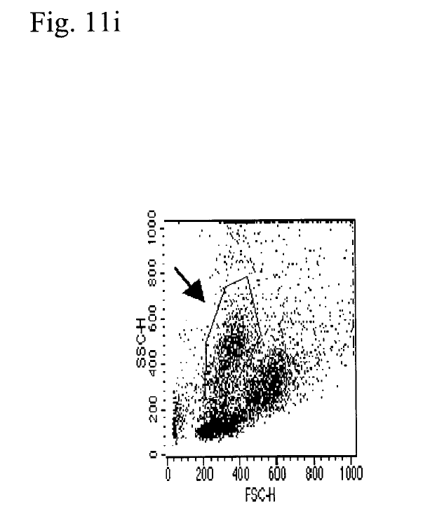

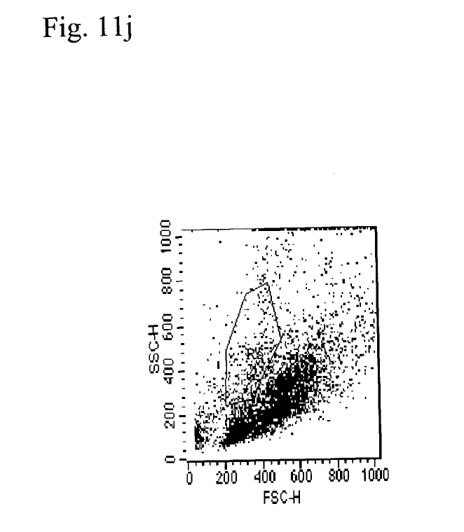

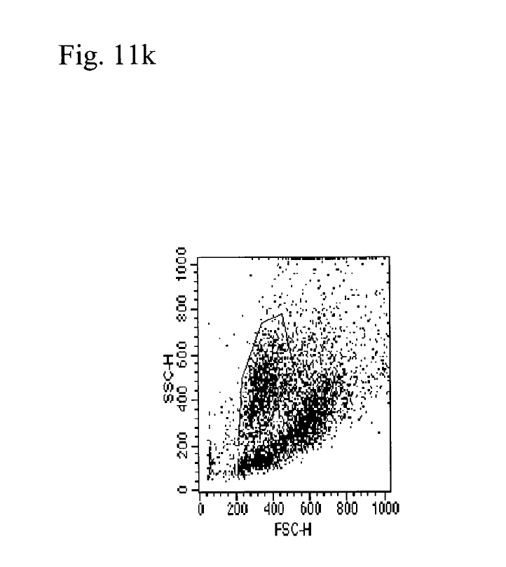

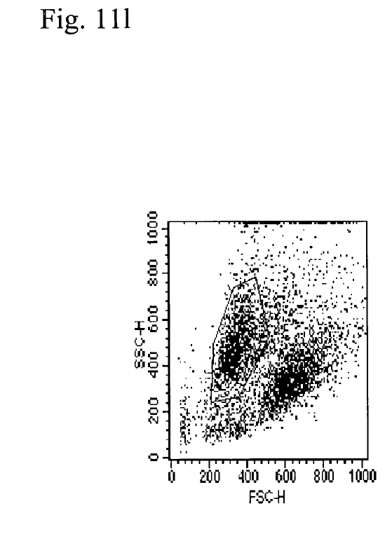

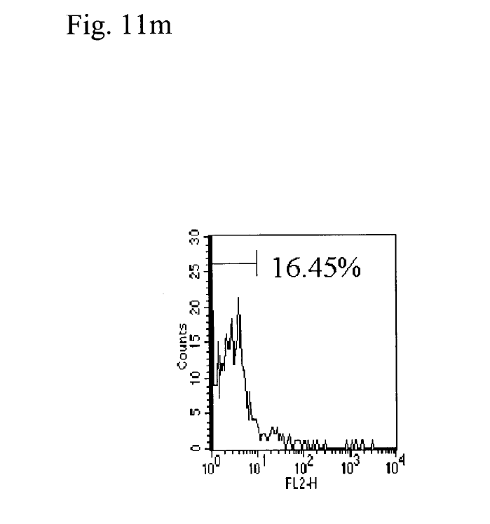

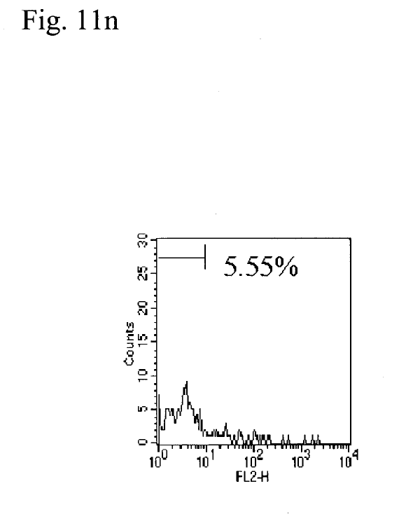

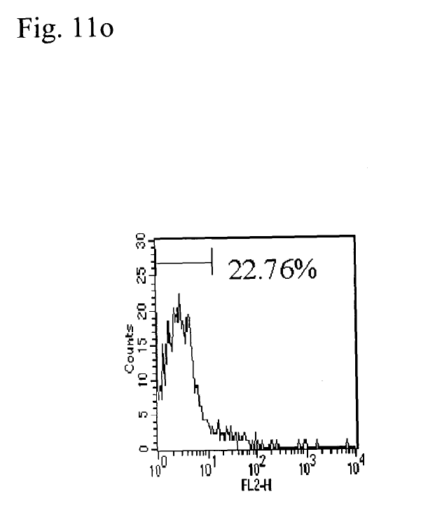

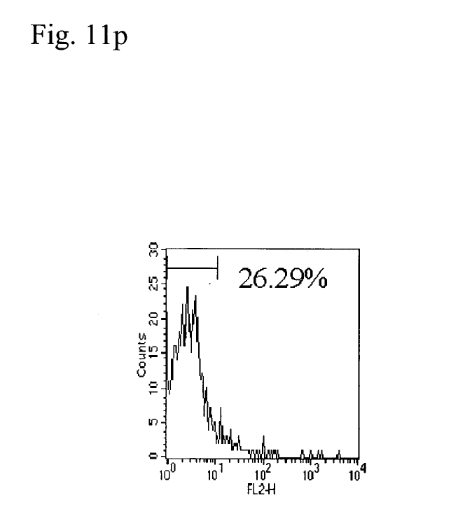

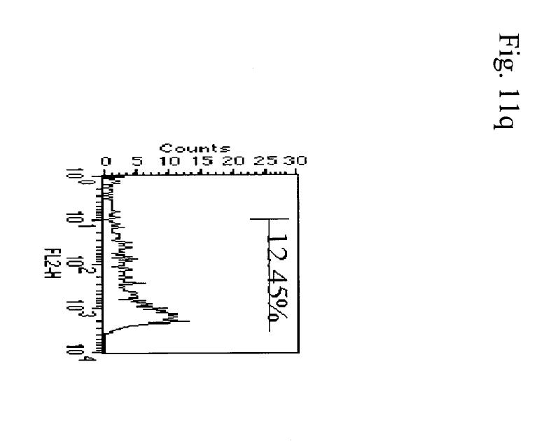

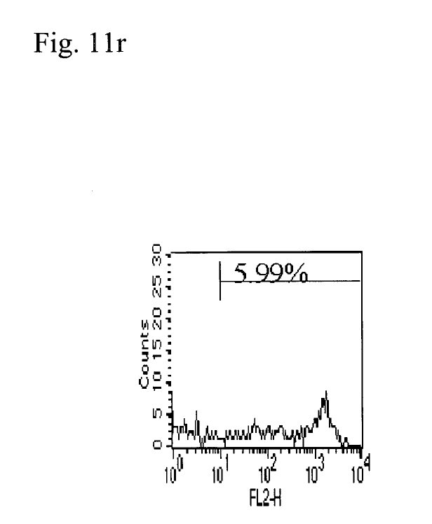

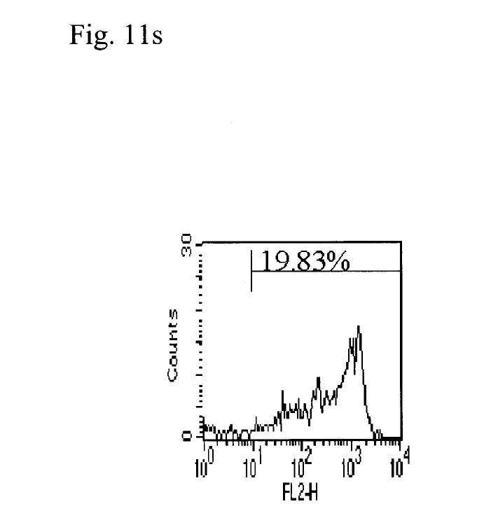

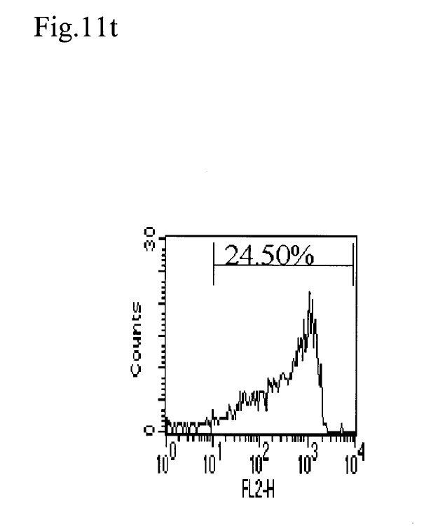

In the result, the percentage of c-Kit positive cells (from femur bone marrow which account for 17.8% of total bone marrow) in irradiated mice was at 19.52% on day 16 (FIG. 11f). In PG101-1 treated mice, the number of c-Kit positive cells was higher at 24.87%, suggesting a stimulatory effect of PG101-1 on the development of c-Kit positive progenitor cells 16 (FIG. 11g). A similar observation was made in G-CSF treated mice. It is worth noting that the 25% increase of c-Kit positive cells in PG101-1 treated mice as compared with control mice is very significant because the percentage of c-Kit positive cells is usually maintained at a low level in bone marrow.

On day 24, no further increase of c-Kit expression was detectable in total bone marrow cells. More interestingly, in PG101-1 treated mice, a new cell population emerged on day 24, which was not found on day 16 (compare FIG. 11c with FIG. 11k). This made the overall pattern of dot plots very similar to that of naïve mice (compare FIG. 11i and FIG. 11k).

In irradiated mice treated with PBS, the number of both c-Kit negative and Gr-1 positive was very low (FIG. 11n and 11r). A similar observation was made in G-CSF treated animals (FIG. 11p and 11t). These results indicated that PG101-1 might be involved in efficiently inducing the differentiation of progenitors and further proliferation of the cells committed to granulocyte lineage.

### Experimental Example 12

### Measurement of Cytokine Levels

Effects of PG101-1 on various cytokines were tested in irradiated mice. Mice were irradiated and treated daily with PG101-1 at 10 mg. Levels of the cytokines in the serum were measured using the commercially available kits at day 8, 16, and 24. ELISA kits for TNF-α, IL-1β, IL-6 and GM-CSF were purchased from Endogen (Woburn, Mass.) and levels of the cytokines were measured according to the manufacturer's instruction.

Radiation is known to result in serious dys-regulation of cytokine expression (Neta R and Oppenheim J. J., Blood, 72, pp1093-1095, 1998). And it was reported that IL-1β and IL-6 might be involved in the recovery of blood cells after irradiation (Neta R. et al., J. Exp. Med., 175, pp689-694, 1992; Zeidler C et al., Blood, 80, pp2740-2745, 1992), while GM-CSF might play important roles in proliferation and differentiation of bone marrow cells (Neta R et al., Lymphokine Res., 5, pp105-110, 1986).

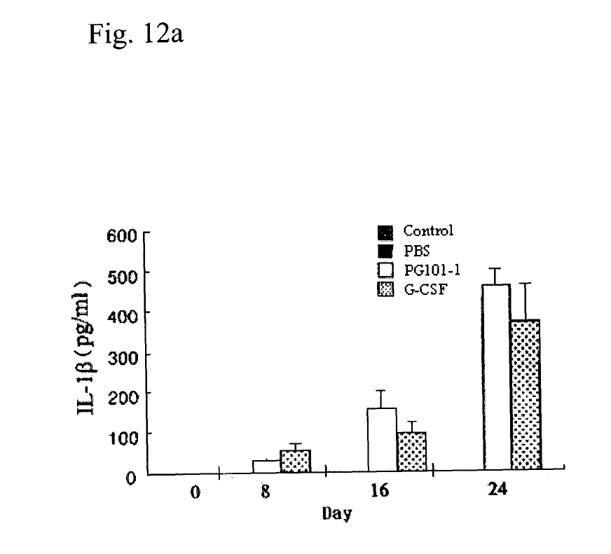

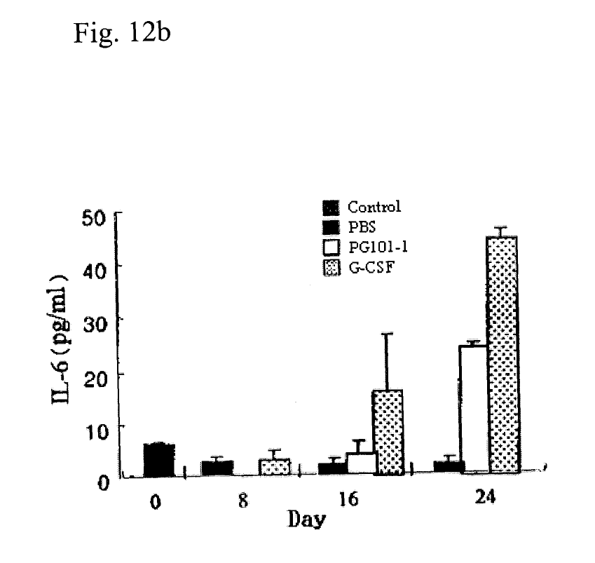

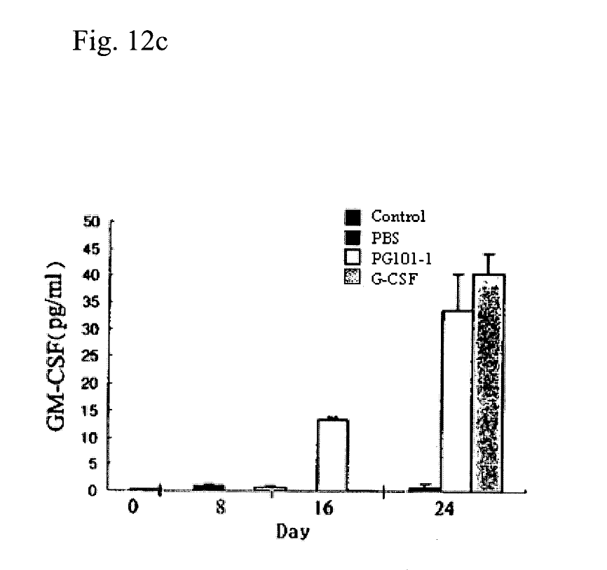

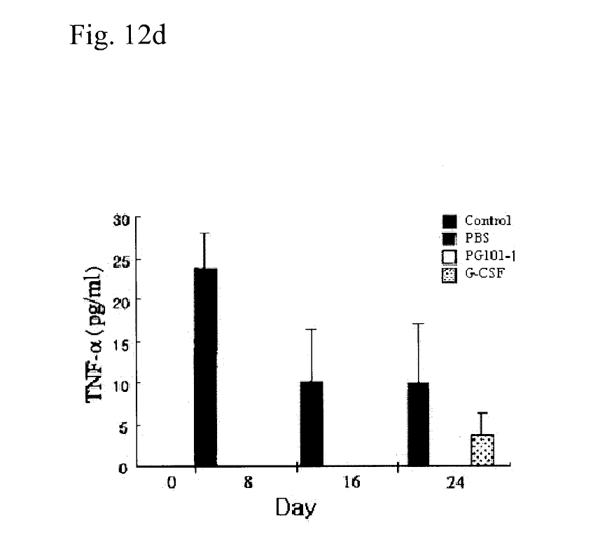

As clearly shown in FIGS. 12a to 12c, levels of IL-1β, IL-6, and GM-CSF were significantly increased in PG101-1 treated mice. On the contrary, the level of TNF-α, whose level had been increased after irradiation, was decreased over time.

These results suggested that PG101-l could increase serum levels of radioprotective cytokines, while decreasing the level of radioinduced TNF-α.

These results suggested that PG101-1 could be used as a very effective BRM in case of damaged immune system.

### Experimental Example 13

### Effect of PG101-1, PG101-2 and Lentinan on the Activation of Human PBMCs

To compare the effect of PG101-1 and that of PG101-2 and lentinan, those samples were treated to PBMCs.

Hereinafter, lentinan means the water extract from fruit body of Lentinus edodes.

PBMCs were obtained from the blood samples of healthy volunteers and isolated by Ficoll-Hypaque (Amersham Pharmacia Biotech AB) gradient centrifugation. Approximately 1×10⁶ cells were incubated with RPMI 1640 medium containing 10% FBS and 10 μg/Ml of PG101-2 at 37° C. under an atmosphere containing 5% CO₂ for 24 hours. And then the levels of cytokines responded to treatment in cultured medium were measured using commercial ELISA microplate reader.

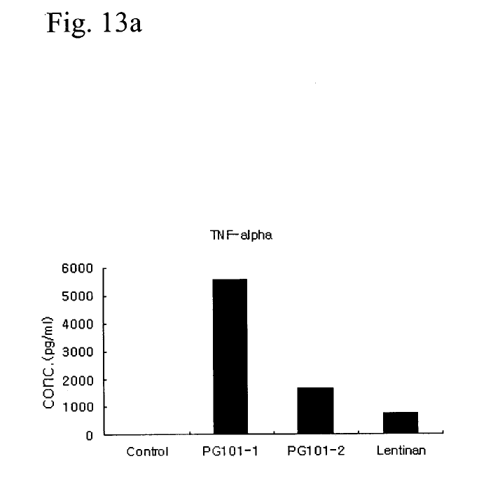

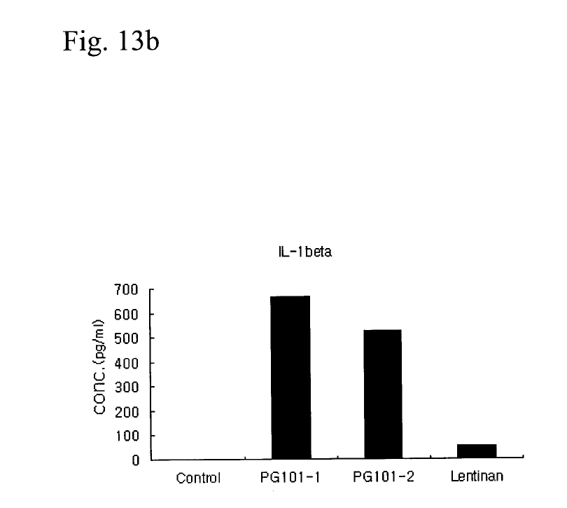

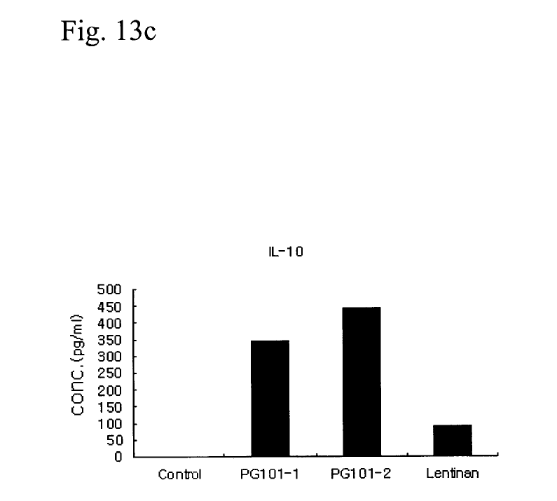

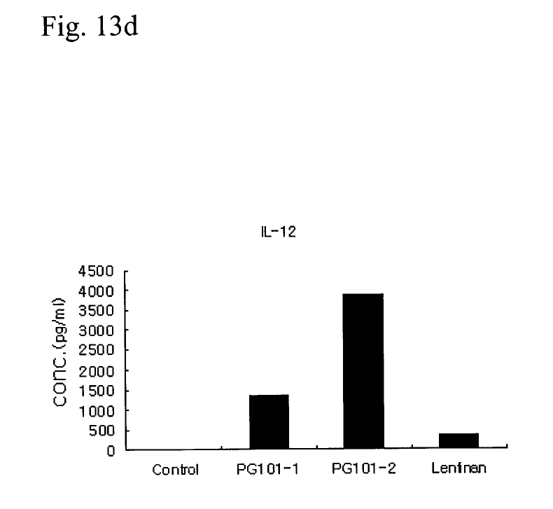

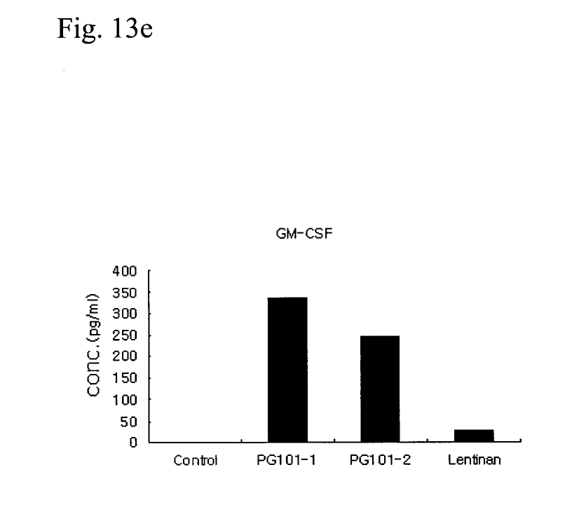

As shown in FIG. 13a to 13e, the level of TNF-α expression by PG101-1 was twice as high as that of PG101-2, however, IL-12 was highly expressed in PG101-2 treated group. IL-1β, IL-10 and GM-CSF were expressed similarly in two groups.

The levels of cytokines in lentinan-treated group were 10 times less than those of PG101-1 or PG101-2 treated group.

Finally, it was confirmed that PG101-1 and PG101-2 were far superior to lentinan in immunological activation.

### Experimental Example 14

### Analysis of Cytokines Activated by PG101-1 in Human PBMCs Using cDNA Chip

Human PBMCs were isolated from blood and treated with PG101-1 under identical experimental method of Experimental Example 13.

RNA sample of PBMCs were extracted using TRIzol (GIBCO BRL) and labeled with fluorescence labeling kit.

Cy3-labeled cDNA was synthesized with RNA from control group treated with PBS and Cy-5-labeled cDNA with RNA from PG101-1 treated group.

The mixture of above two kinds of fluorescence-labeled cDNA was hybridized at 65° C. on cDNA chip (IntelliGene Human Cytokine CHIP Version 2.0, Takara, Japan) and detected the fluorescence using Affymetrix 418 array scanner (Affymetrix, USA).

The value of expression was obtained from control group and experimental group and was calculated by formula 1 belows;

[Formula 1]

Value of expression=each fluorescence of gene spot−background fluorescence

For setting the standard, average activation value of 4 beta-actin gene spots on chip was adjusted with multiplying by a certain number. And activation values of all gene spots were multiplied by the certain number to produce standardized activated fold.

Standardized repressed fold was produced by division of the expression value of control group with the expression value of experimental group and standardization

As the result of Table 5 and 6, 42 kinds of genes were activated by PG101-1 while 28 kinds of genes were repressed among 240 cytokine genes and its related genes.

TABLE 5

| Gene | GeneBank Accession No. | Activated Fold |
|---|---|---|
| I-309 | M57506 | 188.3 |
| IL-6 | X04430 | 69.4 |
| M-CSF | M37435 | 49.2 |
| CCL20 | U64197 | 40.4 |
| GROα | X54489 | 33.7 |
| Inhibin β | J03634 | 31.6 |
| SPP1 | AF052124 | 30.2 |
| IL-1α | M28983 | 27.7 |
| IL-1β | M15330 | 19.8 |
| MIP-2α | M36820 | 16.6 |
| MMP1 | AK024818 | 15.8 |
| | D90145 | 10.0 |
| SCYα3L | Z48482 | 8.8 |
| MMP15 | U56725 | 8.1 |
| Hsp70 | X51602 | 8.1 |
| FLT | X02910 | 5.8 |
| TNF-α | X06374 | 5.4 |
| PDGFα | NM_00230 | 5.1 |
| LIF | M21574 | 5.0 |
| PDGFRα | D90144 | 4.8 |
| MIP-1α | U16261 | 4.6 |
| STM 16 | NM_00171 | 4.3 |
| BMP6 | M54995 | 4.0 |
| B Thrombin | X01057 | 4.0 |
| MIP-1α | J04130 | 2.4 |
| IL2Rα | M26683 | 2.2 |
| MCP-1 | | |

TABLE 6

| Gene | GeneBank Accession No. | Repressed Fold |
|---|---|---|
| CSF-1 | X3663 | −8.9 |
| CCR2α | U95626 | −8.4 |
| IGR1 | AL050337 | −7.2 |
| G-CSFR1 | M59820 | −7.1 |
| Endoglin | NM_00011 | −6.2 |
| CD4 | U47924 | −5.3 |
| TIMP2 | AL110197 | −4.7 |
| CD86 | U04343 | −4.7 |
| IFN−γ induced monokine | X72755 | −3.7 |
| CX3CR1 | U20350 | −3.0 |
| TNF member 13 | AF114012 | −2.8 |
| IL1R2 | X59770 | −2.8 |
| IFN-γR2 | U05875 | −2.8 |
| IL-10Rα | U00672 | −2.7 |
| HLA-DR7 | M16941 | −2.6 |
| TNF member 10 | U37518 | −2.5 |
| CXCR4 | A147204 | −2.5 |
| G-CSFR2β | M59941 | −2.5 |
| TNF member 5 | X68550 | −2.4 |
| HGFR | J02958 | −2.4 |
| Vitronectin R | M14648 | −2.2 |
| IL-10Rβ | Z17727 | −2.2 |
| Heme oxigenase 1 | Z82244 | −2.1 |
| Caspase3 | U13737 | −2.1 |
| DRI3I | M32578 | −2.0 |
| E2D1 | AF020761 | −2.0 |

### Experimental Example 15

### Cytokine Activation by PG101-1 in Northern Blot Analysis

In order to confirm above results of cDNA chip of Experimental Example 14, Northern blot analysis was performed with the RNA samples from PBMCs treated PG101-1 or PBS.

20 μg of RNA was loaded on agarose gel containing formaldehyde and separated in size during electrophoresis. The RNA was transferred on nylon membrane with capillary method. The membrane was baked in 80° C. oven for 2 hours.

RT-PCR was performed to make probes of TNF-α, IL-1 and IL-10. 1 μg of RNA obtained from human PBMCs was reverse transcribed using commercially available RT-PCR kit (Superscript™ II RT, Invitrogen) to make cDNA. Above cDNA was amplified with primers of each gene probe in PCR cycler. The primer sequence of each gene is as follows; TNF-α primer set, 5′-TTGAATTCTTAGTGGTTGCCAGCAC-3′, 5′-GTTCCTCAGCCTCTTCTCCTTCCTG-3′; IL-1β primer set, 5′-TCATCTTTCMCACGCAGGACAGGT-3′, 5′-TCATCTTCAACACGCAGGACAGGT-3′ and IL-10 primer set, 5′-CTGCACCCACTTCCCAGGCMC-3′, 5′-CCCCAGCCCAGAGACMGATAAA-3′.

Probes of respective genes, made by RT-PCR, were radioactively labeled with [³²P]-dCTP using primer labeling kit (Prime-it RmT Random Primer Labeling kit). Nylon membrane adsorbed a labeled probe and RNA was hybridized in order that each probe was to be bound with complementary RNA and bound probes were identified by exposing with X-ray film.

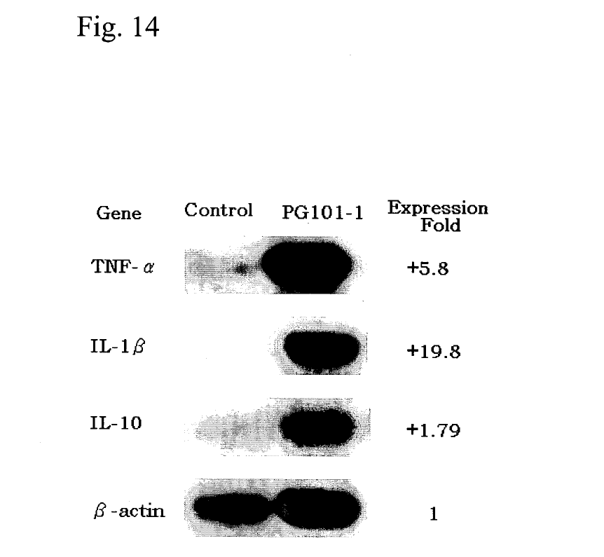

In the result, the gene expression such as activated TNF-α, IL-1β and IL-10 was reconfirmed in Northern blot analysis as the result of cDNA chip of Experimental Example 14 (FIG. 14).

### Experimental Example 16

### Determination of Directly Affected-Cytokine by PG101-1

To find the cytokine directly induced by PG101-1 among various cytokine released by PG101-1, we performed following experiment using the cDNA chip.

Human PBMCs were isolated from blood and maintained as identical experimental method of Experimental Example 13. 10 μg/Me of cyclohexamide, inhibitor of protein synthesis, was added to cultured medium and incubated for 1 hour. And then 100 μg/Ml of PG101-1 was treated for 4 hours. In control, only 10 μg/Ml of cyclohexamide was treated.

As shown in Table 7, it was confirmed that 7 genes were activated; TNF-α was the most activated gene by 14.7 fold and Mip gene such as Mip-1α and IL-18 were activated.

TABLE 7

| Gene | GeneBank Accession No. | Activation Fold | Only PG101-1 |
|---|---|---|---|
| TNF-α | X02910 | 14.7 | 5.8 |
| SCYA3L1 | D90145 | 9.4 | 10 |
| MIP-1α | D90144 | 7.2 | 4.9 |
| MIP-1β | X01057 | 6.5 | 2.5 |
| GRO2 | M36820 | 6.3 | 16.6 |
| IL-8 | M26383 | 3.9 | 1.7 |
| MIP-3α | U64197 | 2.5 | 40 |
| IL-1β | M15330 | 1.3 | 20 |

### Experimental Example 17

Genes confirmed in above Experimental Example 16 were analyzed again using Northern blotting method.

RNA samples were extracted according to the method of above Experimental Example 16 and those were executed in Northern blot analysis with identical method of above Experimental Example 15. In this experiment, probes of IL-8 and Mip-1α were prepared using IL-8 primer set 5′-GACATACTCCAAACCTTTCCA-3′, 5′-ACTGTGAGGTAAGATGGTGGC-3′ and Mip-1α primer set 5′-AGCCTTGGGAAACATGCGT-3′, 5′-CCCTGMCAAAAGCATCCGAT-3′.

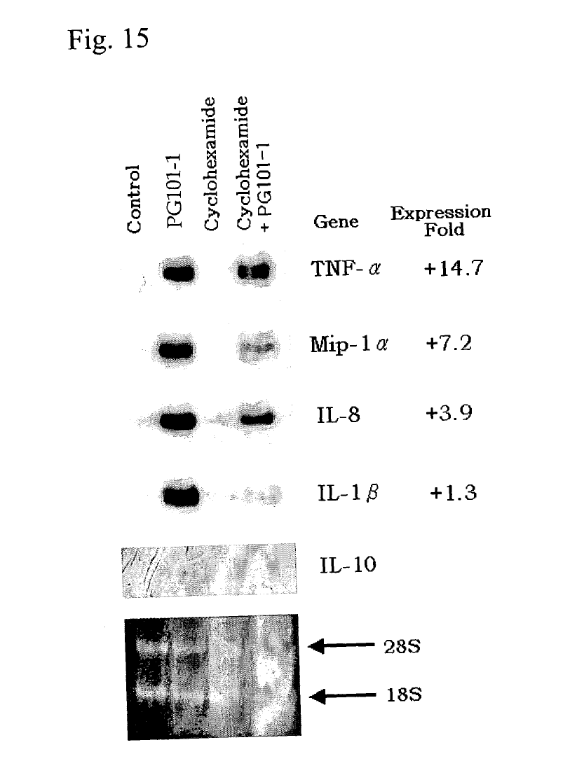

As shown in FIG. 15, TNF-α, Mip-1α and IL-8 showed their genetic expression regardless of presence of cyclohexamide. It proved that PG101-1 directly activated these genes. However, IL-1β gene expression by PG101-1 was decreased when cyclohexamide was added. It was confirmed that IL-1β gene expression was activated not only by PG101-1 directly but also by other cytokines. In case of IL-10, its gene expression was disappeared clearly in presence of cyclohexamide. It could be understood that IL-10 was activated by other expressed cytokines.

### Experiment Example 18

### Toxicity Test

In order to examine the cytotoxicity of PG101-1 obtained in the Example 1, the experiment was performed as follows.

Methods

The acute toxicity on SPF Sprague-Dawley rats (Biogenomics), having its mean body weight of 108.3˜126.0, was performed using PG101-1. Each group consisting of 5 rats was administrated orally with 8000 mg/kg of PG101-1 and observed for 14 days. This test was carried out in compliance with the Testing Guidelines for Safety Evaluation of Drugs (Notification No. 1999-61) issued by Korea Food and Drug Administration and the Good Laboratory Practice Regulations for Non-clinical Laboratory Studies (Notification No. 2000-63) issued by Korea Food and Drug Administration and OECD Principles of Good Laboratory Practice.

Results

There were no treatment-related effects on mortality, clinical signs, body weight changes and gross findings in any group or either gender by using 8000 mg/kg of PG101-1. These results suggested that the compounds prepared in the present invention were potent and safe.

Hereinafter, the formulating methods and kinds of excipients will be described, but the present invention is not limited to them. The representative preparation examples were described as follows.

Preparation of powder

| | |
|---|---|
| Dried powder of Example 1 | 50 mg |
| Lactose | 100 mg |
| Talc | 10 mg |

Powder preparation was prepared by mixing above components and filling sealed package.

Preparation of tablet

| | |
|---|---|
| Dried powder of Example 1 | 50 mg |
| Corn Starch | 100 mg |
| Lactose | 100 mg |
| Magnesium Stearate | 2 mg |

Tablet preparation was prepared by mixing above components and entabletting.

Preparation of capsule

| | |
|---|---|
| Dried powder of Example 1 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of injection

| | |
|---|---|
| Dried powder of Example 1 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| Dried powder of Example 1 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of health food

| | 1000 mg | |
|---|---|---|
| Extract of Example 1 | optimum amount | |
| Vitamin mixture | | |
| Vitamin A acetate | 70 | μg |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in may ways. Such variations are not to be regarded as a departure from the spirit and scope of the present invention.

Preparation of health beverage

| | | |
|---|---|---|
| Extract of Example 1 | 1000 | mg |
| Citric acid | 1000 | mg |
| Oligosaccharide | 100 | g |
| Apricot concentration | 2 | g |
| Taurine | 1 | g |
| Distilled water | 900 | Ml |

Health beverage preparation was prepared by dissolving active component, mixing, stirred at 85° C. for 1 hour, filtered and then filling all the components in 1000 Ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

In the Specificati n:

Please amend the specification as shown:

Please delete the paragraph on page 26, lines 2-16, and replace it with the following paragraph:

For NF-κB, U937 cells were cultured at 5×10⁵/Ml for 16-20 hours and stimulated with PG101-1 as indicated time. Nuclear extracts were prepared using by the treated or not treated cells with PG101-1 (Phares et al., J. Virol., 66, pp7490-7498, 1992). The oligonucleotide probe corresponding with −86, −116 region of HIV LTR promoter known to be inter-reacted with NF—εB, was reacted together with labeled ₃₂P. The reaction mixture containing 3 μg of poly(dI-dC), 0.3 ng of double stranded [₃₂P]-labeled oligonucleotide NF-κB probe (5′-ATCCTCCGCTGGGGACTTTCCAGGGAGGA-3′) (SEQ ID NO: 1) and 3 μg of nuclear extracts in binding buffer (10 mM Tris-HCl, pH 7.5, 50 mM NaCl, 1 mM DTT, 5% glycerol, 2 μg of bovine serum albumin, 3 mM GTP) were prepared to the extent that its final volume was made to 20 μl. After incubation for 20 min at room temperature, the reaction solution was analyzed on a low-ionic strength 4% polyacrylamide gel using by running buffer (6.7 mM Tris-HCl, pH 7.5, 3.3 mM sodium acetate, pH 7.0, 1 mM EDTA, pH 7.5). The specificity of the retarded complex was confirmed by competition test with 40-fold excess of cold wild type or mutant oligonucleotide (5′-GATCCTCCGCTCTCGACTTTCCAGGGAGGA-3′) (SEQ ID NO: 2).

Please delete the paragraph on page 37, line 8 to page 38, line 7, and replace it with the following paragraph:

RT-PCR was performed to make probes of TNF-α, IL-10 and IL-10. 1 μg of RNA obtained from human PBMCs was reverse transcribed using commercially available RT-38 PCR kit (Superscript™ II RT, Invitrogen) to make cDNA. Above cDNA was amplified with primers of each gene probe in PCR cycler. The primer sequence of each gene is as follows; TNF-α primer set, 5′-TTGAATTCTTAGTGGTTGCCAGCAC-3′ (SEQ ID NO: 3), 5′-GTTCCTCAGCCTCTTCTCCTTCCTG-3′(SEO ID NO: 4); IL-1β primer set, 5′-TCATCTTTCAACACGCAGGACAGGT-3′ (SEQ ID NO: 5), 5′-TCATCTTCAACACGCAGGACAGGT-3′ (SEQ ID NO: 6) and IL-10 primer set, 5′-CTGCACCCACTTCCCAGGCAAC-3′ (SEQ ID NO: 7), 5′-CCCCAGCCCAGAGACAAGATAAA-3′ (SEQ ID NO: 8).

Please delete the paragraph on page 39, lines 4-9, and replace it with the following paragraph:

RNA samples were extracted according to the method of above Experimental Example 16 and those were executed in Northern blot analysis with identical method of above Experimental Example 15. In this experiment, probes of IL-8 and Mip-1α were prepared using IL-8 primer set 5′-GACATACTCCAAACCTTTCCA-3′ (SEQ ID NO: 9), 5′-ACTGTGAGGTAAGATGGTGGC-3′ (SEQ ID NO: 10) and Mip-1α primer set 5′-AGCCTTGGGAAACATGCGT-3′ (SEQ ID NO: 11), 5′-CCCTGAACAAAAGCATCCGAT-3′ (SEQ ID NO: 12).

1. An extract of Lentinus lepideus showing immune enhancing and immune modulating activity prepared by the method comprising the steps of: (a) mixing mycelia of Lentinus lepideus, which are incubated in liquid medium, collected from cultured media using filter, washed with distilled water and dried, or fruit bodies of Lentinus lepideus with distilled water, heating the mixture to extract the active ingredients and centrifuging to obtain the supernatant and (b) concentrating the above supernatant and drying to obtain inventive brown extract comprising heteroglycan and monosaccharides such as glucose, mannose and galactose.

2. The extract according to claim 1 wherein the Lentinus lepideus is the naturally occurring or artificially cultivated product thereof.

3. A preparation method of said extract as set forth in claim 1; characterized in that: (a) mycelia of Lentinus lepideus stored on agar medium are incubated at the temperature ranging from 15 to 35° C., the speed ranging from 30 to 200 rpm, for the period ranging from 1 to 20 days in liquid medium; (b) and then mycelia are collected from cultured media using filter; (c) the above mycelia are washed with distilled water, dried and mixed with 5 to 20-fold volume of distilled water, lower alcohols such as methanol, ethanol and the like, or the mixtures thereof, treated with hot water at the temperature ranging from 50 to 100° C. for the period ranging from 1 to 48 hours with 2 to 5 times and centrifuged to obtain the supernatant; (d) the above supernatant is concentrated to obtain said extract.

4. A pharmaceutical composition comprising said extract as set forth in claim 1 as an active ingredient in an amount effective to prevent infectious disease and treat patients suffered from the disorder of immune system together with a pharmaceutically acceptable carrier.

5. The composition according to claim 4 wherein said immune disorder is caused by immune deficiency or suppressed immunity.

6. The composition according to claim 4 wherein said patient is suffered from suppressed immunity due to anti-cancer therapy such as chemotherapy and radiotherapy.

7. The composition according to claim 4 wherein said patient is suffered from AIDS or cancer.

8. The composition according to claim 4 wherein said extract is contained about 0.01 to 95 w/w % based on the total weight of the composition.

9. The composition according to claim 4 wherein said composition is provided in an acceptable carrier as powder, granule, tablet, capsule, aqueous medicine or injection.

10. An immunomodulating drug comprising said extract as set forth in claim 1 as an active ingredient in an amount effective to treat patients suffered from the disorder of immune system together with a pharmaceutically acceptable carrier.

11. A method for treating a patient afflicted with infectious diseases comprising administering to mammal an effective amount of said extract as set forth in claim 1 together with a pharmaceutically acceptable carrier thereof.

12. A method of enhancing immunity in a mammal or human comprising administering to said mammal or human an effective amount of said extract as set forth in claim 1 and pharmaceutically acceptable carrier thereof.

13. A feed or feed additive comprising said extract as set forth in claim 1 as an active ingredient for the livestock, to enhance immunity against infectious diseases.

14. The feed or feed additive according to claim 13, wherein said extract is contained 0.01 to 95 w/w % of feed to enhance immunity against infectious diseases.

15. A method for treating a livestock afflicted with infectious disease, which comprises administering feed and a feed additive containing said extract as set forth in claim 1 to enhance immunity of the livestock.

16. A health food comprising above extract as an active ingredient in an amount effective to enhancing immunity, together with a sitologically acceptable additive.

17. The health food according to claim 16 wherein said health food is provided as powder, granule, tablet, capsule or beverage type.

18. The health food according to claim 16 wherein said ratio of additive is ranging from 1 to 20 w/w % per 100 w/w %.

19. A method for preventing infectious disease, which comprises administering health food containing said extract as set forth in claim 1 to enhance immunity.
